(19) United States
(12) Patent Application Publication
(10) Pub. No.: US 2023/0329587 A1
(43) Pub. Date: Oct. 19, 2023

(54) System And Method For Assessing Neuro Muscular Disorder By Generating Biomarkers From The Analysis Of Gait

(71) Applicant: LifeGait, Inc., Wilmington, NC (US)

(72) Inventors: Christopher D. NEWTON, Greenwood Village, CO (US); Kenneth ZEIGER, Carolina Beach, NC (US); Mark WILLIAMS, Wilmington, NC (US)

(21) Appl. No.: 18/302,275

(22) Filed: Apr. 18, 2023

Related U.S. Application Data
(60) Provisional application No. 63/332,000, filed on Apr. 18, 2022.

(51) Int. Cl. A61B 5/11; A61B 5/00

(52) U.S. Cl. CPC A61B 5/112; A61B 5/7264; A61B 5/6898; A61B 2562/0219

(57) ABSTRACT

Systems and methods for analyzing and representing human gait using motion sensor data may include collecting raw motion sensor data from a mobile device held on or near the user's sternum. The mobile device (or another device) may be configured to generate an information structure representation based on the raw motion sensor data, determine a gait cycle based on the information structure representation, identify gait events in the gait cycle, extract gait biomarkers based on the identified gait events, and determine a diagnosis based on the extracted biomarkers.

### RELATED APPLICATIONS

This application claims the benefit of priority to U.S. Provisional Application No. 63/332,000, entitled ‘System And Method For Assessing Neuro Muscular Disorder By Generating Biomarkers From The Analysis Of Gait’ filed Apr. 18, 2022, the entire contents of which are hereby incorporated by reference for all purposes.

### BACKGROUND

The peripheral nervous system (PNS) is an important part of the nervous system that functions as a communication network between the central nervous system (CNS) and the rest of the body. The CNS, which includes the brain and spinal cord, processes and interprets information from the PNS, which in turn carries out the necessary responses. The PNS includes nerves and ganglia (clusters of nerve cell bodies) that extend from the spinal cord and brain to various body parts, such as muscles, skin, and internal organs. It can be further divided into the somatic nervous system and the autonomic nervous system. The somatic nervous system controls voluntary movements and relays sensory information. The autonomic nervous system manages involuntary functions, such as heartbeat, digestion, and respiration. The autonomic nervous system can be further subdivided into sympathetic and parasympathetic systems, which work together to maintain the body's internal balance or homeostasis.

The PNS plays a vital role in transmitting information, enabling the body to respond and adapt to its environment. Neuromuscular disorders include a broad spectrum of medical conditions that impact the functioning of muscles and the PNS. The origin of these disorders may be attributed to various factors, such as genetic mutations, autoimmune responses, infections, or exposure to specific toxins. Some well-known examples of neuromuscular disorders include Parkinson's disease, Alzheimer's disease, and muscular dystrophy. People affected by these disorders often experience symptoms that include muscle weakness, impaired movement, muscle stiffness, and difficulties with coordination or balance. In some instances, these conditions may also lead to respiratory, cognitive, or sensory complications. The severity and progression of neuromuscular disorders may differ significantly between individuals, which could make diagnosing and treating these conditions challenging.

Neuromuscular disorders affect millions of people worldwide, significantly influencing their overall well-being and daily activities. The complex nature of these disorders, combined with their diverse symptoms, makes diagnosis and monitoring a challenging task. Consequently, there is a need for new and improved methods, systems, and devices that are faster, cheaper, more convenient, and more effective for assessing, diagnosing, and/or monitoring these and other disorders.

### SUMMARY

Various aspects include methods of representing human gait to diagnose health conditions, which may include collecting raw motion sensor data from a mobile device (in which the raw motion sensor data may be recorded in three-dimensional format), generating an information structure representation of the collected raw motion sensor data, determining a gait cycle based on the generated information structure representation, identifying gait events in the gait cycle, extracting gait biomarkers based on the identified gait events, and determining a diagnosis based on the extracted gait biomarkers. In some aspects, generating the information structure representation of the collected raw motion sensor data may include generating a biokinetographic (BKG) waveform information structure based on the raw motion sensor data.

In some aspects, collecting motion sensor data from the mobile device may include collecting sensor data from a sensor in the mobile device that may be positioned on a specific part of a body of a mobile device user as the mobile device user walks a closed course. In some aspects, collecting motion sensor data from the mobile device may include passively collecting sensor data from a sensor in the mobile device. Some aspects may further include reorienting the raw motion sensor data based on the orientation of the mobile device during the collection of the raw motion sensor data to ensure consistent alignment of an axis of three-dimensional sensor data.

Some aspects may further include using a neural network to identify gait patterns and classify gait abnormalities, in which determining the diagnosis based on the extracted gait biomarkers may include determining the diagnosis based on the extracted gait biomarkers, identified gait patterns, and classified gait abnormalities. Some aspects may further include collecting additional sensor data from one or more of a magnetometer, a pressure sensor, a light sensor, a microphone, or an infrared sensor, in which determining the diagnosis based on the extracted gait biomarkers comprises determining the diagnosis based on the extracted gait biomarkers and the additional sensor data.

In some aspects, the mobile device may be a smartphone. In some aspects, collecting motion sensor data from the mobile device may include collecting the raw motion sensor data from a wearable device held on or near a sternum of a mobile device user, and the operations of generating the information structure representation of the collected raw motion sensor data, determining the gait cycle based on the generated information structure representation, identifying the gait events in the gait cycle, extracting the gait biomarkers based on the identified gait events, and determining the diagnosis based on the extracted gait biomarkers are performed on at least one of a smartphone or server computing device.

Some aspects may further include sending the raw motion sensor data from the wearable device to the smartphone and sending the raw motion sensor data from the smartphone to the server computing device.

Further aspects may include a computing device (e.g., smartwatch, smartphone, server, etc.) having a processor configured with processor-executable instructions to perform various operations corresponding to the methods discussed above.

Further aspects may include a computing device having various means for performing functions corresponding to the method operations discussed above.

Further aspects may include a non-transitory processor-readable storage medium having stored thereon processor-executable instructions configured to cause a processor to perform various operations corresponding to the method operations discussed above.

### BRIEF DESCRIPTION OF THE DRAWINGS

The accompanying drawings, which are incorporated herein and constitute part of this specification, illustrate exemplary embodiments, and together with the general description given above and the detailed description given below, serve to explain the features of various embodiments.

FIG. 1 is a block diagram illustrating example components of a computing system 102 that could be configured in accordance with some embodiments.

FIG. 2 is a system block diagram illustrating components that may be included in the computing system 102 configured in accordance with various embodiments.

FIGS. 3A and 3B are illustrations that illustrate examples of how walking patterns and gait analysis may be correlated with events that could be used for diagnosing various health conditions in accordance with the various embodiments.

FIGS. 4-10 are process flow diagrams that illustrate methods using a mobile device to represent human gait and diagnose health conditions (e.g., detect a neuromuscular disorder, etc.) in accordance with various embodiments.

FIG. 11 is a block diagram illustrating an example system on chip suitable for implementing the various aspects.

FIG. 12 is a system block diagram illustrating components that could be configured to represent human gait and diagnose health conditions in accordance with various embodiments.

FIG. 13 is a component block diagram of a wearable device suitable for implementing some of the embodiments.

FIG. 14 is a component block diagram of a mobile device suitable for implementing some of the embodiments.

FIG. 15 is a component diagram of an example server suitable for implementing some embodiments.

### DETAILED DESCRIPTION

The various embodiments will be described in detail with reference to the accompanying drawings. Wherever possible, the same reference numbers will be used throughout the drawings to refer to the same or like parts. References made to particular examples and implementations are for illustrative purposes and are not intended to limit the scope of the invention or the claims.

In overview, the various embodiments include methods, and computing systems and devices configured to implement the methods, for assessing disorders (e.g., neuromuscular disorders, etc.) by generating biomarkers from gait analysis. In some embodiments, the computing system (e.g., wearable device, smartphone, server, etc.) may collect data from an accelerometer, generate graphs (or information structure representations) based on the collected data, extract an analog signal from the graphs, condition the analog signal to extract information and biomarkers, convert the analog signal to a digital output, processes and clean the data, and display the information on a visually accessible interface.

Some embodiments may include a computing device (e.g., mobile device, etc.) configured to receive data from a motion sensor (e.g., an accelerometer, gyroscope, etc.) while the user holds or wears the device (or another device containing the motion sensor) and walks a designated distance. The computing device (or another device, such as a remote server, etc.) may process the motion sensor measurements and correlate them to data points related to the user's gait. The computing device may analyze the data points to determine the presence of biomarkers linked to a specific disease state, such as neuromuscular disorders like concussion, Parkinson's, or Alzheimer's. The computing device may be configured to allow for remote monitoring and assessment of the disorders so that it may operate without the supervision of a doctor or medical professional. The computing device may enhance the detection and management of a variety of disorders and provide valuable insights for both individuals and healthcare providers.

The embodiments may improve or enhance the performance or function of computing devices while offering numerous advantages over traditional methods of diagnosing and monitoring disorders. For example, some embodiments may allow users to track their condition using a device they likely already possess (e.g., a smartphone), removing the necessity for extra specialized equipment. In addition, some embodiments may provide remote monitoring capabilities that allow users to evaluate their neuromuscular health without needing to visit a healthcare professional in person. This makes healthcare more accessible for individuals with limited mobility or those residing in isolated locations. Further, by eliminating or minimizing the need for in-person consultations and specialized equipment, the various embodiments may reduce healthcare expenses for both patients and healthcare providers. The embodiments may also enable users to identify neuromuscular symptoms at an early stage, potentially resulting in more effective treatment strategies and enhanced patient outcomes. Further improvements to the performance and functioning of computing devices will be evident from the disclosures herein.

The term ‘computing device’ may be used herein to refer to any one or all of server computing devices, personal computers, laptop computers, ultrabooks, tablet computers, user equipment (UE), smartphones, mobile devices, a medical device or equipment, biometric sensors/devices, wearable devices (smart watches, smart clothing, smart glasses, smart wrist bands, smart jewelry (for example, smart ring, smart bracelet)), internet-of-things (IOT) devices, personal or mobile multi-media players, wireless electronic mail receivers, multimedia Internet enabled cellular telephones, and other similar devices that include a programmable processor for providing the functionality described herein.

The term ‘mobile device’ may be used herein to refer to any one or all of wireless devices, IOT devices, cellular telephones, smartphones, personal or mobile multi-media players, personal data assistants (PDA's), laptop computers, tablet computers, palm-top computers, wireless electronic mail receivers, multimedia Internet enabled cellular telephones, wireless gaming controllers, wearable devices and similar electronic devices which include a programmable processor, a memory and circuitry for sending and/or receiving wireless communication signals. While some embodiments are particularly useful in wireless mobile devices, such as smartphones, the embodiments are generally useful in any electronic device that includes a programable processor suitable for executing software applications.

The terms ‘component,’ ‘system,’ and the like may be used herein to refer to a computer-related entity (e.g., hardware, firmware, a combination of hardware and software, software, software in execution, etc.) that is configured to perform particular operations or functions. For example, a component may be, but is not limited to, a process running on a processor, a processor, an object, an executable, a thread of execution, a program, and/or a computing device. By way of illustration, both an application running on a computing device and the computing device may be referred to as a component. One or more components may reside within a process and/or thread of execution and a component may be localized on one processor or core and/or distributed between two or more processors or cores. In addition, these components may execute from various non-transitory computer-readable media having various instructions and/or data structures stored thereon. Components may communicate by way of local and/or remote processes, function or procedure calls, electronic signals, data packets, memory read/writes, and other known computer, processor, and/or process-related communication methodologies.

The term ‘neural network’ may be used herein to refer to an interconnected group of processing nodes (e.g., neuron models, etc.) that collectively operate as a software application or process that controls a function of a computing device or generates a neural network inference. Individual nodes in a neural network may attempt to emulate biological neurons by receiving input data, performing simple operations on the input data to generate output data, and passing the output data (also called ‘activation’) to the next node in the network. Each node may be associated with a weight value that defines or governs the relationship between input data and activation. The weight values may be determined during a training phase and iteratively updated as data flows through the neural network.

Deep neural networks implement a layered architecture in which the activation of a first layer of nodes becomes an input to a second layer of nodes, the activation of a second layer of nodes becomes an input to a third layer of nodes, and so on. As such, computations in a deep neural network may be distributed over a population of processing nodes that make up a computational chain. Deep neural networks may also include activation functions and sub-functions (e.g., a rectified linear unit that cuts off activations below zero, etc.) between the layers. The first layer of nodes of a deep neural network may be referred to as an input layer. The final layer of nodes may be referred to as an output layer. The layers in-between the input and final layer may be referred to as hidden layers, intermediate layers, or black-box layers.

Each layer in a neural network may have multiple inputs, and thus multiple previous or preceding layers. Said another way, multiple layers may feed into a single layer. For ease of reference, some of the embodiments are described with reference to a single input or single preceding layer. However, it should be understood that the operations disclosed and described in this application may be applied to each of multiple inputs to a layer as well as multiple preceding layers.

The term ‘convolutional neural network’ (CNN) may be used herein to refer to a deep neural network in which the computation in at least one layer is structured as a convolution. A convolutional neural network may also include multiple convolution-based layers, which allows the neural network to employ a very deep hierarchy of layers. In convolutional neural networks, the weighted sum for each output activation is computed based on a batch of inputs, and the same matrices of weights (called ‘filters’) are applied to every output. These networks may also implement a fixed feedforward structure in which all the processing nodes that make up a computational chain are used to process every task, regardless of the inputs. In such feed-forward neural networks, all of the computations are performed as a sequence of operations on the outputs of a previous layer. The final set of operations may generate the overall inference result of the neural network, such as a probability that an image contains a specific object (e.g., a person, cat, watch, edge, etc.) or information indicating that a proposed action should be taken.

The term ‘inference’ may be used herein to refer to a process that is performed at runtime or during the execution of the software application program corresponding to the neural network. Inference may include traversing the processing nodes in the neural network along a forward path to produce one or more values as an overall activation or overall ‘inference result.’

For ease of reference, some embodiments are described using neural networks. However, it should be understood that various embodiments may use any of a variety of machine learning techniques, such as random forest, linear regression, logical regression, support vector machines (SVM), naive bayes, decision trees, etc. As such, nothing in this application should be used to limit the machine learning techniques to neural networks unless expressly recited as such in the claims.

The term ‘biokinetographic’ (BKG) may be used herein to refer to a technique for measuring a person's movements, such as gait or other physical activities, based on the data collected from motion sensors (e.g., accelerometers, gyroscopes, etc.). The terms ‘BKG waveform’ and ‘BKG signature’ may be used herein to refer to an information structure utilized by a computing device to represent motion data (raw motion sensor data) through various formats, such as smooth data arrays, matrix arrays, spectrograms, persistent data flows, connected data points, continuous representations, continuous plots, or other analogous structures or representations. Said another way, the BKG waveform and BKG signature may both be information structure representations of the raw motion sensor data. As an example, the term ‘BKG waveform’ may refer to an information structure used by a computing device to represent changes in motion data over time. This structure may capture variations in acceleration, velocity, or other parameters during distinct phases of movement, and may be utilized to identify patterns and irregularities in an individual's gait or motion. A BKG waveform may also include processed data, various biomarkers (e.g., stance and swing phases), and multiple variables. For instance, a BKG waveform may include variables related to individual steps, such as the duration and magnitude of heel strikes and toe-offs, stride length, cadence, balance, and symmetry. The term ‘BKG signature’ may be used herein to refer to an information structure that characterizes a specific pattern or set of variables that are associated with a particular gait pattern or individual.

The interpretation of human movement is important in various medical specialties, including pediatrics, sports medicine, geriatrics, physical medicine and rehabilitation, neurology, rheumatology, orthopedics, etc. However, motion-based assessments are often limited by subjective clinical impressions from expert observers, and fail to capture the full breadth of sensory information processed during expert evaluations. Accurate and precise motion data may provide deeper insights into an individual's affective, cognitive, and physical performance status, assisting in diagnosing and treating specific dysfunctions such as Parkinson's disease, hemiparesis, cerebellar disease, frontal lobe disease, orthopedic pain, orthopedic injury, motor neuropathy, myopathy, and psychological dysfunction.

The various embodiments include computing devices configured to detect, assess, diagnose, and/or monitor a variety of symptoms, conditions, and disorders. Said another way, the various embodiments may be used for assessing, screening, monitoring, diagnosing, and/or prognosing any of a variety of neurological, orthopedic, cardiovascular, respiratory, psychiatric and geriatric conditions. For ease of reference, some embodiments are discussed with reference to neuromuscular disorders (e.g., Parkinson's disease, Alzheimer's disease, concussion, etc.). However, nothing in this application should be used to limit the claims to neuromuscular disorders unless expressly recited as such in the claims.

Neuromuscular disorders affect millions of people worldwide. These conditions often have significant impacts on an individual's quality of life and may be difficult to diagnose and monitor effectively. The various embodiments include systems, methods, and devices for assessing neuromuscular disorders through gait analysis.

Some embodiments may include a computing system (e.g., mobile device, smartphone, etc.) that is configured to evaluate neuromuscular disorders by generating biomarkers based on the analysis of a person's walking pattern or gait. These biomarkers may provide valuable information about the presence or progression of certain medical conditions.

In some embodiments, the computing system (or a second device) may be equipped with a motion sensor (e.g., an accelerometer, a gyroscope, etc.) that may be worn or held next to a user's chest (or hip, etc.) while they walk a designated distance. The motion sensor may capture hundreds of data points related to the person's walking pattern when the user walks with the device held up against their sternum or the center of their chest. The computing system may analyze these data points to identify biomarkers or determine whether the person exhibits neuromuscular symptoms that are associated with a specific disease or medical condition. The computing system may detect various neuromuscular disorders based on these and other identified biomarkers or symptoms.

In some embodiments, the computing system may be configured to upload the collected data, biomarkers, symptoms and/or analysis results to a cloud-based server. The computing system may be configured to allow for remote monitoring of medical conditions, thereby eliminating the need for direct supervision or monitoring by a doctor or medical professional. This makes the assessment of neuromuscular disorders more convenient and accessible for individuals, as they may use their mobile devices to evaluate their condition without having to visit a healthcare facility.

In some embodiments, the computing system may be configured to implement BKG processes or methods for analyzing an individual's walking patterns or gait to gain insights into their overall health and wellness. The methods may include using one or more sensors (e.g., accelerometer, gyroscope, etc.) to collect data on various aspects of a person's movements, such as heel strike, toe strike, cadence, and balance. The methods may also include generating BKG waveforms, graphs, signatures, images, and/or tracings, and examining the generated elements to identify potential irregularities or deviations in an individual's gait that are indicative of the presence of certain mental and health conditions, such as neuromuscular disorders, frailty, malingering, an increased risk of falls, sobriety, drug discovery, compliance with a drug regimen, etc. Such analysis may be a valuable tool for the early detection, monitoring, screening, and management of these health issues, allowing individuals and their healthcare providers to take appropriate preventive measures and seek necessary treatments. Such analysis may also be a valuable tool for testing for sobriety, drug use, compliance with a drug regimen, etc.

Evaluating neuromuscular disorders through gait analysis in accordance with the embodiments may mark a considerable advancement in the diagnosis and monitoring of neuromuscular conditions. By leveraging powerful sensors that are available in modern computing devices and advanced processing and analysis systems, techniques, or technologies, the embodiments may offer greater accessibility, enhanced cost-efficiency, and increased convenience for individuals affected by neuromuscular disorders.

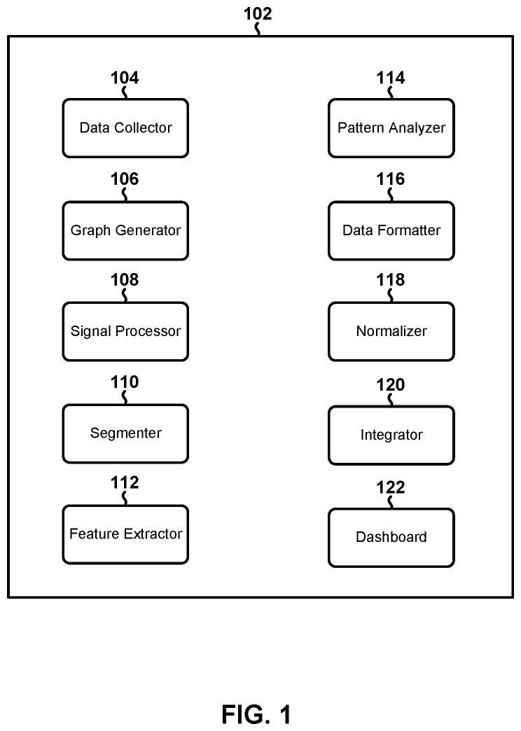

FIG. 1 is block diagram illustrating example components of a computing system 102 that could be configured in accordance with some embodiments. In the example illustrated in FIG. 1, the computing system 102 includes a data collector 104 component, a graph generator 106 component, a signal processor 108 component, a segmenter 110 component, a feature extractor 112 component, a pattern analyzer 114 component, a data formatter 116 component, a normalizer 118 component, an integrator 120 component, and a dashboard 122 component. In various embodiments, the computing system 102 may include any or all of wearable device, smartphone, and server, any or all of which may include any or all of the components 104-122 discussed herein.

The computing system 102 may be configured to produce a clinically accurate indication of a target condition related to an individual's health and wellness. The computing system 102 may allow for early detection and monitoring of various medical conditions, allow individuals to assess and monitor a condition without direct supervision from a doctor or medical professional, allow users to gain insights into their health and wellness, and allow users to track their condition using a device they likely already possess (e.g., a smartphone), thereby removing the necessity for extra specialized equipment and making health assessments more accessible.

The data collector 104 component may be configured to gather information from a sensor positioned on a specific part of the body. For example, the data collector 104 may initiate data collection from an embedded accelerometer, ensure proper placement of the device on the sternum or other suitable body location while the user walks a closed course, and record raw accelerometer data in 3-axis format (x, y, z) as the user walks the closed course.

The data collector 104 may gather information from any of a wide variety of sensors, including accelerometers, ambient light sensors, barometers, blood oxygen saturation (SpO2) sensors, cameras (or other optical sensors or photo optic sensors), chemical sensors, compasses (magnetometers), electrocardiogram (ECG) sensors, force meters, gas content analyzers, Geiger counters, global positioning system (GPS) receivers or other satellite geopositioning sensors, gravimeters, gyroscopes, heart rate sensors, humidity sensors, impact sensors, infrared sensors (e.g., for use in measuring heart rate variability, etc.), LIDAR, microphones, motion sensors, near field communication (NFC) chips, neutron detectors, object detection and ranging sensors, optical sensors, pH sensors, photoplethysmogram (PPG) sensors, pressure sensors, proximity sensors, pedometers, radiation sensors, RADAR, skin temperature sensors, strain sensors, stress meters, temperature sensors, touch sensors, ultrasonic sensors, vibration sensors, and other perception sensors, or any other sensor found in modern mobile devices such as smartphones and smartwatches.

In some embodiments, any or all of the sensors (e.g., accelerometers, gyroscopes, magnetometers, etc.) may be three-dimensional sensors that may measure and record data in all three dimensions (x, y, and z axes) of physical space and/or detect changes in position, orientation, or motion. In some embodiments, motion signals and data from each sensor may be sampled at various frequencies, ranging from 20 Hz to 2500 Hz, including all values and sub-ranges within this range. In some embodiments, the computing device may determine the number, types and/or configurations of sensors selected for data collection based on the purpose of the assessment (diagnosis, monitoring, or predicting disability).

The graph generator 106 component may be configured to use the collected data to generate graphs or other information structures (or BKG waveforms) that represent the user's gait.

The signal processor 108 may perform various signal processing operations on these graphs (or BKG waveforms) to extract information that characterizes the gait in a suitable analog signal format. These operations may include noise reduction for more accurate and consistent data analysis. In some embodiments, the signal processor 108 component may work in conjunction with a normalization component (e.g., normalizer 118) to reorientate the data to correct for the device's orientation during data collection before analyzing the data to detect key biomarkers, and scale and normalize the data to ensure that the next component receives input in a standardized format (standardized input). The signal processor 108 may regenerate to update the graphs (or BKG waveforms) based on the results of the processing operations.

In some embodiments, these components may reorientate the data to correct for the device's orientation during data collection. Such processing may allow for more accurate and consistent data analysis and for the accommodation of different devices and orientations. The signal processor 108 may regenerate to update the graphs (or BKG waveforms) based on the results of the signal processing operations.

The segmenter 110 may identify key biomarkers in the gait cycle (e.g., heel strikes, toe-offs, etc.), determine gait phases (e.g., stance phase, swing phase, double stance phase, etc.), and isolate specific events, such as the turnaround in the test and steps before and after the turnaround. The segmenter 110 may segment the data into gait cycles or individual steps or strides. The computing device may apply the segmented data to various components that implement mathematical equations or statistical methods to calculate key features, such as stride length, cadence, step duration, and foot strike patterns.

The feature extractor 112 may calculate displacement, velocity, and acceleration using double integration and trapezoidal integration, extract additional variables from the raw data (e.g., harmonic ratios, center of mass displacement, etc.), and organize the extracted variables into domains (e.g., force, stride, balance, symmetry, etc.). In some embodiments, the computing device may apply the extracted variables to a neural network (e.g., a CNN, etc.) and/or use other machine learning techniques (including both supervised and unsupervised learning) for data cleansing and pattern recognition. In some embodiments, the computing device may examine different axes, zero crossings, and intersections between axes to determine transitions between gait phases. By identifying these key biomarkers, the computing system 102 may extract additional variables from the raw data and organize them into the domains.

In some embodiments, the computing device may be configured to reorient and scale the data in a first phase, and identify key biomarkers, such as heel strikes and toe-offs, in a second phase. In some embodiments, the second phase may include additional processing to ensure data quality, which may involve checking the number of steps taken and normalizing the data using stride lengths. Displacement and velocity information may also be obtained after detecting heel strikes. The computing device may examine the user's center of mass, lateral movement, and forward movement to assess gait stability. This information may be used to extract additional variables, which may then be organized into four main domains, such as a force domain, stride domain, balance domain, and symmetry domain.

Each domain may include a handful of variables that provide insights into the user's gait. Examples of variables include Striking Force, Pushing Force, Forward Power, Side Power, Vertical Power, Stride Time, Stance Phase, Swing Phase, Double Stance, Side to Side Sway, Sway (Front to Back), Support Variability, Power Consistency, Gait Smoothness, Forward Sway Symmetry, Side Sway Symmetry, and Side-To-Side Movement Symmetry. The identified biomarkers may be used to extract the additional variables, such as displacement of the center of mass, stride times, and the harmonic ratio. The device may segment each gait cycle and analyze rhythmicity and smoothness within individual cycles (rather than in the entire signal). Other variables, such as displacement and power, may be extracted by examining the raw amplitudes, peaks, and extrema in the signal. Such comprehensive analysis of the user's gait allows the computing device to derive valuable insights that may be used to accurately determine and characterize the user's walking patterns and health conditions.

The feature extractor 112 may include raw accelerometer data through components that extract key features of the gait cycle (e.g., heel strike, toe strike, toe-off moments, etc.). For example, the computing device may determine acceleration over time during walking and identify the force exerted when the heel hits the ground, followed by the toe strike, and then the pushing force as the foot swings. These biomarkers may be determined based on a BKG waveform that corresponds with the user's gait cycle.

Thus, the computing device may derive key events from the data, extracting numerous biomarkers or variables. Some variables may not rely on specific heel strike biomarkers and are considered nonlinear, as they pertain to the frequency domain rather than individual heel strikes. The computing device may examine the signal as a whole and decompose it into the frequency domain to analyze rhythmicity and smoothness.

The pattern analyzer 114 may perform various machine learning and pattern recognition operations. For example, the pattern analyzer 114 may train supervised and unsupervised machine learning models on the collected data, use the trained models to classify gait patterns, detect anomalies, and predict potential conditions, and refine the models through cross-validation and collaboration between cross-functional teams. By training machine learning models on the collected data, the system may classify gait patterns, detect anomalies, and predict potential conditions.

In some embodiments, the computing device may use neural networks or machine learning algorithms to recognize patterns and classify gait abnormalities. For example, the computing device may use supervised learning techniques in which a trained model is applied to the processed data to identify specific gait-related biomarkers indicative of a certain health condition. Additionally, the computing device may implement time-frequency analysis methods, such as Fast Fourier Transform (FFT) or wavelet analysis, to identify and quantify periodic components in the motion sensor data, revealing gait characteristics not easily discernible in the time domain. By applying multiple analytical techniques, the computing device may extract a wealth of information from the motion sensor data and diagnose or monitor neuromuscular disorders more effectively.

In some embodiments, the computing device may use machine learning algorithms to recognize patterns and classify gait abnormalities by leveraging large datasets containing labeled examples of both healthy and abnormal gaits. In the training phase, the computing device may process the motion sensor data and extract relevant features, such as stride length, cadence, step duration, and foot strike patterns. The computing device may input these extracted features to a neural network or machine learning algorithm that learns to identify patterns associated with specific gait abnormalities by comparing the input data to the labeled examples in the dataset.

In some embodiments, the computing device may use supervised learning components, such as support vector machines, decision trees, or neural networks, to build a classification model. The supervised learning components may adjust their internal parameters during the training process to minimize or reduce the difference between their predictions and the actual labels in the dataset. This allows the model to generalize and recognize similar patterns in new and unseen data. Once the model is trained, the computing device may apply the model to BKG waveform or to the processed motion sensor data of an individual's gait. The model may then output a predicted label or a probability distribution over potential labels, indicating the presence or absence of specific gait abnormalities. This approach may enable the early detection and classification of gait-related issues and/or may allow the computing device to generate a more accurate diagnosis for neuromuscular disorders.

In some embodiments, the computing device may be configured to use a neural network to process and transform input data (e.g., motion sensor data, such as stride length, cadence, step duration, and foot strike patterns) through a series of mathematical operations. For example, the computing device may apply the motion sensor data to the input layer of the neural network. During the training phase, the neural network learns to identify patterns associated with specific gait abnormalities by adjusting the weights and biases of its connections using a labeled dataset containing examples of both healthy and abnormal gaits. The learning process may include iteratively updating the weights and biases to minimize the difference between the predicted labels and the actual labels in the dataset. This is maybe achieved through a process called backpropagation and an optimization algorithm like gradient descent. Once the neural network is trained, it may be used to analyze an individual's processed motion sensor data. The computing device may apply input data to the input layer to cause the output layer to generate a predicted label or a probability distribution over potential labels, indicating the presence or absence of specific gait abnormalities. This approach may enable the early detection and classification of gait-related issues and/or may allow the computing device to generate a more accurate diagnosis for neuromuscular disorders. The use of neural networks, particularly deep learning techniques like convolutional neural networks (CNNs) and recurrent neural networks (RNNs), may lead to more accurate and robust recognition of complex gait patterns, further improving the effectiveness of the diagnosis.

The data formatter 116 may format the data to account for variations in data formats from different sensors or devices.

The normalizer 118 may calculate normative data and risk levels. For example, the normalizer 118 may compare the extracted variables to normative data based on factors such as age, gender, and previous tests. The normalizer 118 may determine the risk of specific conditions (e.g., concussions, etc.) based on individual risk levels and deviations from the norm. In some embodiments, the normalizer 118 may represent the results on a scale indicating low to high risk, such as a visual scale from green to red or a numerical scale from 1 to 10.

The integrator 120 may integrate additional health metrics and sensor data into the results. For example, the integrator 120 may collect data from other sensors (e.g., magnetometers, pressure sensors, light sensors, etc.), measure additional health metrics (e.g., sleep, hydration, heart rate variability, etc.), and combine the gait analysis data with the additional health metrics to evaluate overall health and recovery curves.

The dashboard 122 may visualize and present the results on an electronic display. For example, the dashboard 122 may generate graphs and visualizations based on the extracted variables and calculated risk levels, display the results on a user-friendly interface or dashboard, and allow users to interact with the data and visualize their progress over time.

The computing system 102 may also store and archive the data. For example, the computing system 102 may save the raw data, extracted variables, calculated results, graphs or BKG waveforms in a secure database, maintain data privacy and security according to regulatory guidelines, and provide users options to export or share their data for further analysis or consultation with healthcare professionals.

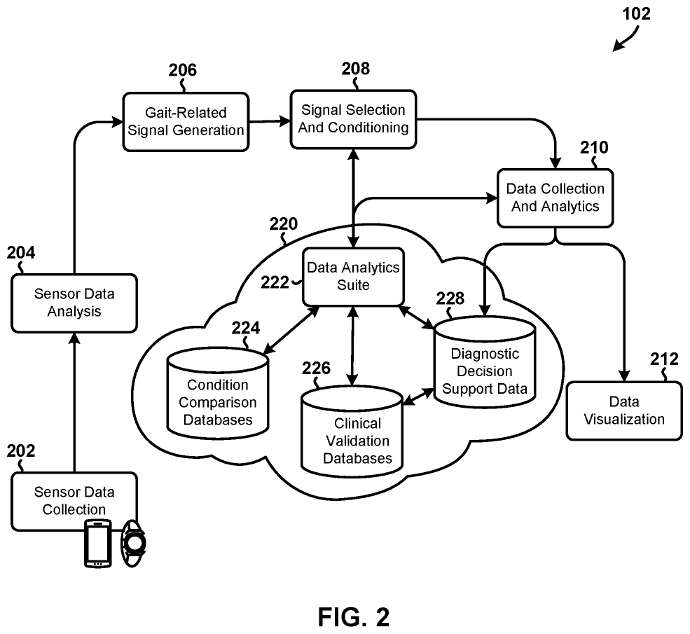

FIG. 2 is a system block diagram illustrating components that may be included in the computing system 102 configured in accordance with various embodiments. In the example illustrated in FIG. 2, the system 200 includes a sensor data collection 202 component, sensor data analysis 204 component, gait-related signal generation 206 component, signal selection and conditioning 208 component, data collection and analytics 210 component, data visualization 212 component, analytical system 220 component, data analytics suite 222 component, condition comparison database 224 component, clinical validation database 226 component, and diagnostic decision support data databases 228.

It should be understood that any or all of the components 202-228 may be, may be included in, and/or may work in conjunction with any or all of the components 104-122 discussed above with reference to FIG. 1. It should also be understood that any or all of the components 104-122, 202-228 may be included in one or more computing devices. For example, in some embodiments, all of the components 104-122, 202-228 may be included in a mobile device (e.g., smartphone, etc.). As another example, in some embodiments, the sensor data collection 202 and sensor data analysis 204 components may be included in a mobile device and the other components 206-228 may be included in a server computing device. As yet another example, in some embodiments, the sensor data collection 202 component may include in a wearable device (smartwatch, etc.), the sensor data analysis 204 component may be included in a smartphone, and the other components 206-228 may be included in a server computing device, which may be deployed in a cloud network.

In some embodiments, computing system 102 may be configured to perform data collection via components 202 and 204, graph creation via component 206, signal conditioning and digital conversion via components 208-210, and digital output presentation on the dashboard via component 212.

The sensor data collection 202 component may be configured to use a motion or mobility sensor, such as an accelerometer or gyroscope in a mobile device, to gather information about walking patterns and data associated with different elements of gait. In some embodiments, the information may be collected from an embedded accelerometer (e.g., tri-axial piezo-resistive accelerometer, etc.) that is held or placed on a specific body part (e.g., sternum, sacrum, waist, ankles, etc.) while walking a closed course. The sensors may measure accelerations related to changes in velocity and gravitational acceleration based on direction and magnitude. For example, when a person walks and changes direction (turns around) in the closed course, their body experiences changes in velocity, which may be detected by the sensors. The sensors may also measure the constant force of gravity acting on the body as the user completes the closed course. By analyzing the direction and magnitude of these accelerations, the computing device may characterize an individual's movements and identify patterns or irregularities in their motion.

In some embodiments, the sensor data collection 202 component may collect additional data from any sensor discussed herein or commonly found in modern smartphones. The computing device may use the additional data to enhance data quality, to measure additional aspects of the user's gait, or to better characterize or analyze the user's gait cycle.

The sensor data collection 202 component may record and/or send the information to the sensor data analysis 204 component, which may process or forward the data to the gait-related signal generation 206 component. The sensor data analysis 204 and/or gait-related signal generation 206 component may process and analyze the data to identify elements of the person's gait. These elements may include aspects such as heel strike, toe strike, toe lift-off, and cadence.

The gait-related signal generation 206 component may generate graphs (or information structure representations) representing the user's gait, process the graphs to extract an analog signal characterizing the gait, and generate a BKG waveform information structure that represents or characterizes the extracted analog signal. In some embodiments, the gait-related signal generation 206 component may also filter data to remove outliers and segment steps or events (e.g., to offer a better understanding of gait patterns, etc.), examine velocity and displacement to extract biomarkers that determine the full range of motion across each axis, identify the relative position of the peaks within the gait cycle (which may be indicators for various conditions), and perform other similar operations. The gait-related signal generation 206 component may send the extracted analog signal to the signal selection and conditioning 208 component for further processing.

The signal selection and conditioning 208 may condition the analog signal to extract features and relevant information, apply signal processing algorithms to analyze raw accelerometer data and generate a digital signal based on the extracted analog signal.

The signal selection and conditioning 208 may work in conjunction with the analytical system 220 component and/or data collection and analytics 210 component to numerous operations to analyze gait data. For example, the components (e.g., 208, 210, 220, etc.) may determine transitions between gait phases by examining factors such as zero crossings of specific axes and intersections between axes. These transitions may help identify when a heel strike occurs or when one gait cycle phase ends, and another begins. Distances between heel strikes may also be considered, providing insights into walking or running and the accuracy of identified heel strikes. The components may check for specific conditions in candidate data points, identify candidate points, review them for accuracy, and examine nearby peaks that might represent the same event. Such comprehensive analysis may provide valuable insights into an individual's gait, allowing the system 200 to better characterize the walking patterns and overall health.

In some embodiments, the components may identify key biomarkers and extract additional variables.

In some embodiments, the components may calculate displacement using double integration and trapezoidal integration to determine speed and distance traveled from raw accelerometer data.

In some embodiments, the components may calculate normative data, and compare the normative data to various factors (e.g., age, gender, previous tests, etc.) to determine the risk of specific conditions.

In some embodiments, the components may use a neural network and/or machine learning to generate probability scores for certain conditions.

In some embodiments, the components may perform feature detection and segmentation operations on raw data and process the computed parameters using machine learning techniques for data cleansing.

In some embodiments, the components may examine various additional health metrics, such as sleep and hydration, which play a role in recovery. In some embodiments, the components may integrate health metrics and biomarkers to evaluate overall health and recovery curves.

In some embodiments, the components may use location data and/or elevation information to determine the impact of the user's elevations on the user's gait or biomarkers or overall health and recovery curves. By using location data and elevation information, the computing device may consider or account for the impact of walking at different elevations on gait patterns and how it relates to biomarkers. This may add another layer of analysis for better determining the individual's gait and overall health.

In some embodiments, the components may determine whether there are any biomarkers or signs of health issues related to the person's gait. These biomarkers/signs may be clinically accurate indicators of a target condition, which may include neuromuscular disorders such as Parkinson's disease, Alzheimer's disease, or concussion.

The data visualization 212 component may display the information on an electronic display.

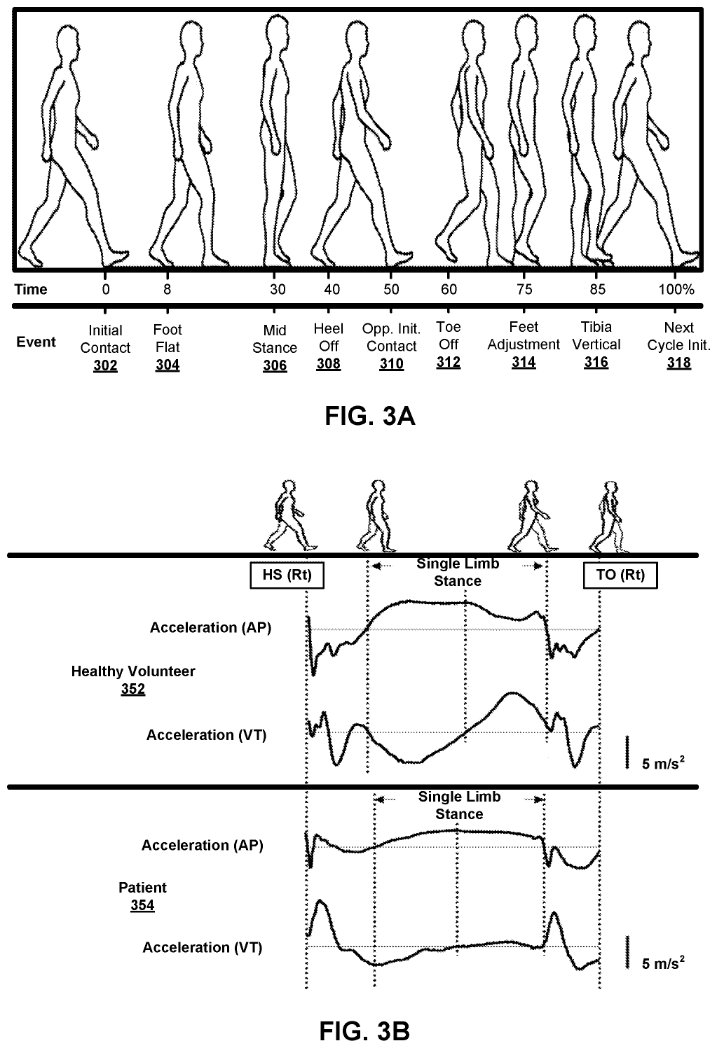

FIGS. 3A and 3B illustrate examples of how walking patterns and gait analysis may be correlated with events that could be used for diagnosing various health conditions in accordance with the various embodiments. Generally, human walking is achieved through a technique known as the double pendulum. As one moves forward, the leg lifting off the ground swings from the hip, constituting the first pendulum. Upon striking the ground with the heel and rolling to the toe, the leg's motion may be described as an inverted pendulum. The two legs' movements may be synchronized to ensure continuous contact with the ground by at least one foot. The calf muscles contract, elevating the body's center of mass and storing potential energy. Subsequently, gravity pulls the body forward and downward onto the other leg, converting the potential energy into kinetic energy. In the various embodiments, these and other motions and data may be captured and represented in various different ways.

In the example illustrated in FIG. 3A, the computing device associates various stages in a single human gait cycle with an event and a time or percentage within the gait cycle. The events include initial contact 302, foot flat 304, mid stance 306, heel off 308, opposite initial contact 310 (or heel strike), toe off 312 (or toe strike), feet adjustment 314, tibia vertical 316, and next cycle initial 318. Each of these events occurs at a certain time (or percent) within the gait cycle.

In the example illustrated in FIG. 3B, the computing device associates two different types of collected acceleration data with various events in a single human gait cycle, including a right foot heel strike (HS (Rt)) and a right foot toe strike (TS (Rt)). In some embodiments, the computing device may be configured to graph the scalar sums of acceleration from each of a plurality of sensors over time (e.g., to generate BKG waveform information structures, etc.). The computing device may compare the information collected (and graphed) from a patient 354 to information collected (and graphed) from a healthy volunteer 352 to identify, for example, potential weaknesses in a person's gait that may indicate frailty or an increased risk of falls or other health concerns. By recognizing such symptoms and risks, individuals may take preventive measures and seek appropriate treatment.

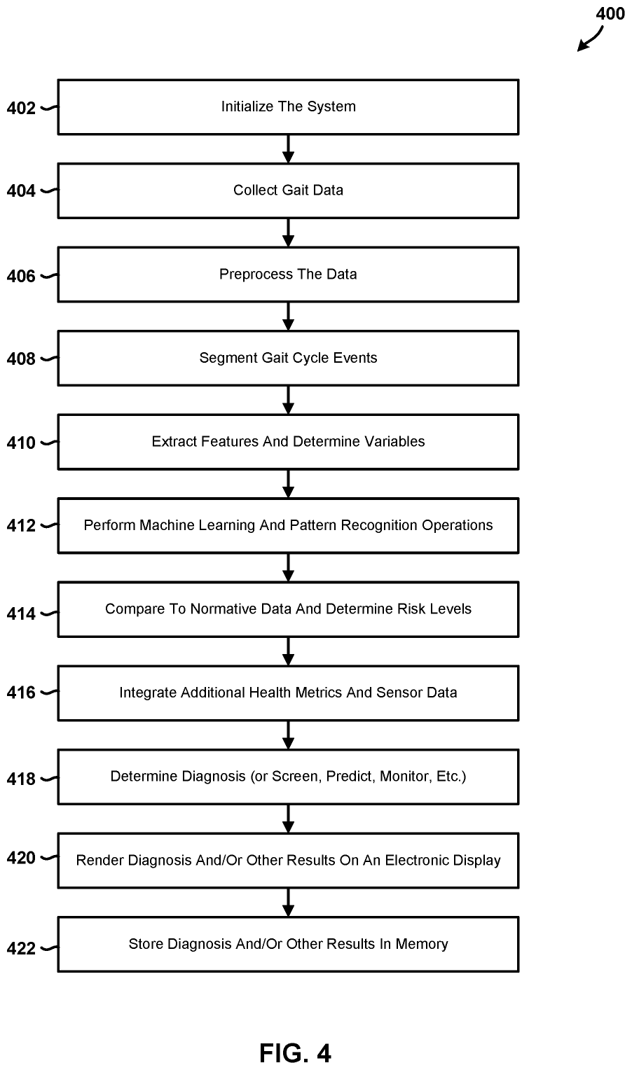

FIG. 4 illustrates a method 400 of detecting a neuromuscular disorder on a mobile device in accordance with some embodiments. All or portions of method 400 may be performed by a processor in a mobile computing device.

In block 402, the mobile computing device may initialize the system. For example, the computing device may load required libraries for data processing, machine learning, and signal analysis, set up connections with the wearable device, phone, or standalone accelerometer, create data storage and archival system, or perform other similar operations.

In block 404, the mobile computing device may collect gait data. For example, the computing device may commence collecting data from the accelerometer, ensure proper placement of the device on the sternum or other suitable body location, and record raw accelerometer data in 3-axis format (x, y, z).

In block 406, the mobile computing device may preprocess the data. For example, the computing device may apply noise reduction techniques, such as low-pass filtering, to smooth the raw data. The computing device may also reorient the data to ensure consistency in the axis alignment, regardless of device orientation. The computing device may also normalize and scale the data to account for variations in data formats from different devices.

In block 408, the mobile computing device may identify and segment gait cycle events from the preprocessed data. For example, the computing device may identify key biomarkers in the gait cycle, such as heel strikes and toe-offs. The computing device may determine gait phases such as the stance phase, swing phase, and double stance phase. The computing device may isolate specific events, such as the turnaround in the test and steps before and after the turnaround.

In block 410, the mobile computing device may extract features and determine variables based on the segmented data. For example, the computing device may determine displacement, velocity, and acceleration using double integration and trapezoidal integration. The computing device may extract additional variables from the raw data, such as harmonic ratios and center of mass displacement. The computing device may organize the extracted variables into domains (e.g., force, stride, balance, symmetry, etc.).

In block 412, the mobile computing device may perform machine learning and pattern recognition, which may include training supervised and unsupervised machine learning models on the collected data, using the trained models to classify gait patterns, detect anomalies, and predict potential conditions, and refine the models through cross-validation and collaboration.

In block 414, the mobile computing device may determine normative data and risk levels. For example, the computing device may compare the extracted variables to normative data based on factors such as age, gender, and previous tests. The computing device may determine the risk of specific conditions, such as concussions, based on individual risk levels and deviations from the norm.

In block 416, the mobile computing device may integrate additional health metrics and sensor data into the results. For example, the computing device may collect data from other sensors, such as magnetometers, pressure sensors, and light sensors. The computing device may measure additional health metrics, such as sleep, hydration, and heart rate variability. The computing device may combine gait analysis data with the additional health metrics to evaluate overall health and recovery curves.

In block 418, the mobile computing device may determine a diagnosis based on the integrated and combined data (including extracted variables and calculated risk levels).

In block 420, the mobile computing device may render the diagnosis and/or other results on an electronic display. For example, the computing device may generate graphs and visualizations based on the extracted variables and calculated risk levels, display the results on a user-friendly interface or dashboard, and allow users to interact with the data and visualize their progress over time.

In block 422, the mobile computing device may store and/or archive the data in memory. For example, the computing device may save the raw data, the extracted variables, and the calculated results in a secure database.

In some embodiments, the computing device may be configured to use data collected from one or more sensors to detect one or more types of motion on one or more body parts (e.g., head, arm, trunk, waist, leg, etc.). In some embodiments, the sensors may simultaneously monitor multiple body parts' movements.

In some embodiments, the computing device may be configured to generate a BKG waveform information structure that represents velocities and accelerations of specific body parts such as the head, trunk, and extremities over time. The computing device may use the generated BKG waveforms to identify movement patterns indicative of particular health conditions or illnesses.

In some embodiments, the computing device may be configured to use the collected sensor data to generate variables suitable for motion analysis. Examples of such variables include a position variable, an orientation variable, a velocity variable, an acceleration variable, a jerk variable, a pulse variable, and/or a torque variable.

In some embodiments, the computing device may be configured to perform motion analysis operations based on the generated variables. In some embodiments, the computing device may incorporate numerous factors that could influence motion or provide contextual information for the collected data in the motion analysis operations. These factors may include ambient factors such as temperature, humidity, and lighting; biological factors such as heart rate and body temperature; physiological factors, including emotional state, cognition, and relative physical performance. By using such contextual information, the computing device may interpret motion data more accurately and provide a more comprehensive analysis of a subject's movements, considering the impact of both internal and external factors.

In some embodiments, the computing device may be configured to use various graphing and analysis techniques to compare gaits and diagnose conditions. For example, the computing device may generate BKG waveforms or graphs by plotting the sum of vector magnitudes (or scalar values) of acceleration from each sensor over time. These BKG waveforms or graphs may represent the motion patterns of an individual, which may include or may be influenced by movement vector components. The movement vector components may be elements of a motion vector that represent the changes in position, orientation, and/or velocity of a body part in three-dimensional space (or expressed along the three primary axes). The computing device may use movement vector components to quantify and analyze various aspects of human motion. The movement vector components may be affected by various factors, including neuromuscular factors like stroke, peripheral neuropathy, or foot drop; mechanical anatomical factors such as previous hip fracture, osteoarthritis, or amputation; and psycho-behavioral conditions like anxiety or depression. These factors may alter aspects of human motion, causing changes in the movement vector components and, consequently, the BKG waveforms or graphs. By analyzing the BKG waveforms or graphs and their underlying movement vector components, the computing device may identify and interpret the effects of these influencing factors on human motion. The computing device may generate more accurate comparisons of gaits and the potential diagnosis of various conditions based on the observed motion patterns and identified effects of the influencing factors.

In some embodiments, the computing device may be configured to perform various operations to analyze the BKG waveform or graph. In some embodiments, the computing device may focus on three key domains: movement biomechanics, energy expenditure, and navigational skill. Such analyses may help identify, evaluate, or account for factors that may influence an individual's motion. For movement biomechanics, the computing device may examine aspects of the movement cycle and other biomechanical elements, which may be determined from the BKG waveform, graph, or other information structure representation, for better modeling or understanding of the individual's motion patterns. The computing device may quantify energy expenditure by analyzing step rate, step rate variability, the magnitude of the Fast Fourier Transformation, and the size or intensity of acceleration values represented on the vertical axis (ordinate) of the BKG waveform, among other factors. For navigational skill, the computing device may assess parameters such as the variability of the ambulatory axis (the path or trajectory followed by a person while walking), the uniformity of Fast Fourier Transformation patterns, and the individual's turning efficiency when changing direction.

In some embodiments, the computing device may be configured to use combinations of biomechanical, energy conservation, and navigational patterns associated with specific clinical states to diagnose various conditions. Such comprehensive analysis may provide valuable insights regarding a person's overall health and mobility.

In some embodiments, the computing device may be configured to generate a BKG signature, which may be a unique representation of an individual's walking pattern or body movement patterns. The BKG signature may be as distinct and individualized as a fingerprint, capturing the specific characteristics of each person's movement.

In some embodiments, the computing device may be configured to perform systematic analysis of BKG waveforms/graphs to quantify various clinical, environmental, motivational, mental, and mechanical elements related to human movement. The computing device may superimpose these elements on the BKG signature to analyze changes in movement (e.g., a change in gait caused by a sprained ankle, etc.). The computing device may analyze the movement signatures for early identification of pre-disease pathways, to generate an objective measure of health or disability, and/or to monitor the effects of treatments.

In some embodiments, the computing device may be configured to collect data from a person or individual over an extended period and generate profiles that track changes in the individual's movements as they age. These profiles may offer valuable insights into movement patterns and performance, facilitating the analysis of age-specific trends and the development of benchmarks for assessing human movement across different age groups. In addition, the computing device may use these profiles to establish norms and indices for human movement and performance. Deviations from these norms could serve as early warning indicators of functional changes before disabilities become permanent or evident through traditional evaluations.

In some embodiments, the computing device may be configured to compare an individual's profile with standard movement patterns to identify focal abnormalities (e.g., irregularities that are localized or confined to a single body part, etc.), which may suggest neurological impairment, arthritis, vascular insufficiency, previous trauma, etc. Numerous other clinical conditions, including neurological, orthopedic, cardiovascular, respiratory, psychiatric, and geriatric syndromes, may be characterized by movement abnormalities. Examples of neurological conditions include stroke, Parkinson's disease, Alzheimer's disease, low pressure hydrocephalus, normal pressure hydrocephalus (NPH), Huntington's disease, demyelinating disease, cerebellar disease, peripheral neuropathy, radiculopathy, autonomic dysfunction, and visual impairment. Examples of orthopedic conditions include low back pain, painful hip, painful knee, painful foot or ankle, and amputation. Examples of cardiovascular diseases include heart disease, peripheral vascular, respiratory disease, and chronic lung disease. Examples of psychiatric illnesses include depression, anxiety, fear, and delirium. Examples of geriatric syndromes may include dizziness, falling, failure to thrive (frailty), etc. In some embodiments, the computing device may be configured to perform a systematic analysis of BKG waveforms to identify critical, characteristic, and defining features of these conditions, with each clinical condition having a unique signature from the BKG.

In some embodiments, the BKG process may include a user walking with their device having a motion sensor (e.g., an accelerometer, a gyroscope, etc.), such as their smartphone, collection of raw motion sensor data (e.g., raw accelerometer data, raw gyroscope data, etc.), application of algorithms to the data to at least in part generate BKG data, determination of quantifiable indicators of the user's motion (e.g., gait), statistical analysis, and determination of biomarkers for the user's condition, such as biomarkers (e.g., neuro muscular symptoms) for a disease state, such as a concussion, Parkinson's and/or Alzheimer's.

In should be noted that BKG waveform of raw data may only represent the mechanical aspects of gait. Other biomarkers, such as range of motion and non-linear biomarkers (e.g., FFT analysis, harmonic ratio, etc.), may not be visible in the BKG waveform itself. However, the computing device may derive these biomarkers from the data points of the motion sensor data.

The diagrams and descriptions provided throughout this discussion, including those related to BKG analysis and example biomarkers, serve merely as illustrations to explain various aspects of the embodiments. These examples are not meant to be limiting in any way. In other words, while BKG analysis is highlighted, it is not the only method that may be used for diagnosing health conditions. In various embodiments, alternative or additional determinations and analyses may be employed to diagnose a condition. For instance, raw motion sensor data may be used to determine other biomarkers, generate new data, or perform statistical analysis. The raw motion sensor data, which may include unprocessed measurements collected by sensors such as accelerometers and gyroscopes, may provide valuable insights about an individual's walking patterns or gait. Accelerometers may measure changes in speed or movement, while gyroscopes may detect changes in angular orientation. These measurements may be analyzed by a computing device to determine various biomarkers that are measurable indicators of specific health conditions.

To extract new insights or generate additional data points relevant to diagnosing or monitoring specific health issues, the computing device may apply the raw motion sensor data to various components. That is, the computing device may derive various markers from the data points of motion sensor data by processing and analyzing the raw measurements obtained from the sensors. For example, the computing device may compute the range of motion in a person's joints by examining the angular orientation changes captured by gyroscopes or calculate the stride length and cadence using the speed and movement data from accelerometers. Additionally, the computing device may perform advanced techniques, such as FFT analysis or harmonic ratio calculations, to identify non-linear markers associated with gait patterns. By analyzing the motion sensor data in these several ways, the computing device may derive a range of markers that may be used to assess an individual's health, diagnose neuromuscular disorders, determine sobriety or compliance with a drug regime, monitor their progress during treatment or rehabilitation, etc.

In some embodiments, the computing device may perform statistical analysis on the raw motion sensor data to identify trends, correlations, or anomalies that might be indicative of underlying health problems. This could involve comparing an individual's data to a larger dataset of healthy individuals or those with known conditions. Advanced statistical techniques, such as regression analysis or machine learning algorithms, may be employed to model and predict health outcomes.

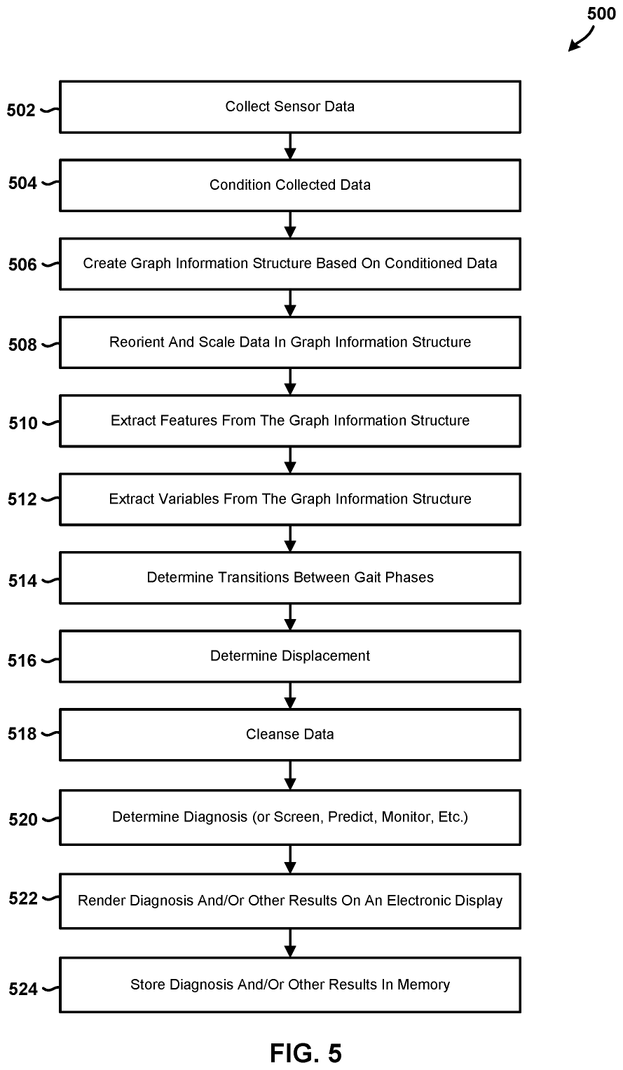

FIG. 5 illustrates another method 500 of detecting a neuromuscular disorder on a mobile device in accordance with some embodiments. All or portions of method 500 may be performed by a processor in a mobile computing device.

In block 502, the mobile computing device may collect sensor data. For example, the computing device may receive data from an embedded accelerometer while the mobile computing device is placed around the sternum area or held next to the user's sternum to measure body sway during walking. The computing device may also receive the sensor data from a wearable device, such as a smartwatch.

In blocks 504 and 506, the mobile computing device may condition the collected data and generate a graph data structure (or other information structure representation) based on the collected data. The computing device may also extract an analog signal structure from the graph and condition the analog signal to extract further information. Since the extracted signal is a digital representation of analog structure that could potentially have an unlimited number of biomarkers, the computing device may convert it to a digital output more suitable for evaluation.

In block 508, the mobile computing device may reorient and scale data in graph information structure. The computing device may detect the device's orientation during data collection and reorient the axis in data if needed before detecting key biomarkers. The computing device may also process the data processing to account for variations in how different devices (e.g., Android and Apple devices, etc.) send data, scaling and normalizing it to ensure that the algorithm receives standardized input.

In block 510, the mobile computing device may apply signal processing to extract features from the graph information structure. For example, the computing device may perform signal processing on the data to extract valuable insights into the user's gait. The raw accelerometer data may be run through algorithms that extract key features of the gait cycle, such as heel strike, toe strike, and toe-off moments. The computing device may determine acceleration over time during walking and identify the forces exerted when the heel hits the ground, followed by the toe strike, and then the pushing force as the foot swings. These biomarkers may be created through the graph or a BKG waveform that corresponds with the gait cycle. The computing device may also determine additional biomarkers from the BKG waveform and/or collected sensor information, such as the stance phase (heel strike to toe-off), swing phase (toe-off to heel strike), and double stance phase (when both feet are on the ground).

In block 512, the mobile computing device may extract variables from the graph information structure. For example, the computing device may extract additional variables organized into domains. The computing device may also use key biomarkers identified in block 510 (e.g., heel strikes and toe-offs, etc.) to extract additional variables. These variables may include the displacement of the center of mass, stride times, and the harmonic ratio. The computing device may also segment each gait cycle and analyze rhythmicity within individual cycles (as opposed to analyzing the entire signal). The computing device may also extract other variables, such as displacement and power, by examining the raw amplitudes, peaks, and extrema in the signal.

In block 514, the mobile computing device may determine transitions between gait phases. For example, the computing device may examine gait data to identify transitions between gait phases by examining factors such as zero crossings of specific axes, intersections between axes, and whether one axis is above or below zero. These transitions may be used to determine when a heel strike occurred or when one phase of the gait cycle ends and another begins.

In block 516, the mobile computing device may determine displacement. For example, the computing device may calculate displacement by employing double integration and trapezoidal integration techniques. These methods may be used to determine speed and distance traveled from the raw accelerometer data.

In block 518, the mobile computing device may cleanse the data. For example, the computing device may use machine learning techniques for data processing and cleansing to ensure that the data is of high quality. These techniques help identify and remove outliers, ensuring the accuracy and quality of the data. In some embodiments, the computing device may perform quality flagging operations to verify that the collected data is accurate and representative of the individual's gait.

In block 520, the mobile computing device may determine a diagnosis based on the integrated and combined data (including extracted variables and calculated risk levels).

In block 522, the mobile computing device may render the diagnosis and/or other results on an electronic display. For example, the computing device may generate graphs and visualizations based on the extracted variables and calculated risk levels, display the results on a user-friendly interface or dashboard, and allow users to interact with the data and visualize their progress over time.

In block 524, the mobile computing device may store and/or archive the data in memory. For example, the computing device may save the raw data, the extracted variables, diagnosis, and the calculated results in a secure database.

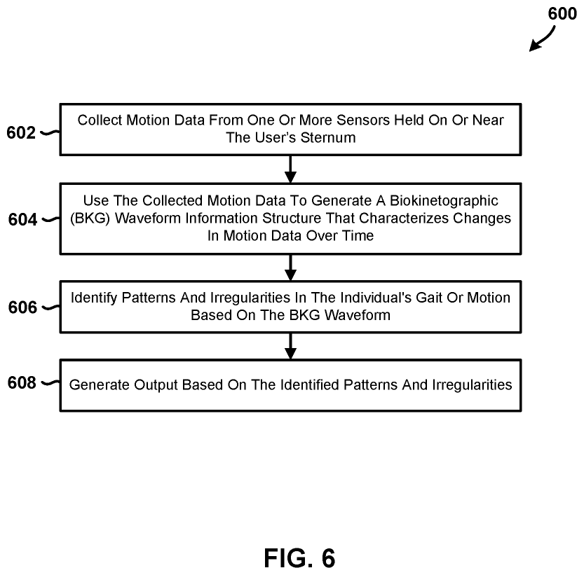

FIG. 6 illustrates a method 600 of representing human gait to diagnose health conditions in accordance with some embodiments. All or portions of method 600 may be performed by a processor in a mobile computing device.

FIGS. 6-10 illustrate methods 600, 700, 800, 900, 1000 of representing human gait to diagnose health conditions in accordance with some embodiments. All or portions of each of methods 600, 700, 800, 900, 1000 may be performed by a processor in a mobile computing device.

With reference to FIG. 6, in block 602, the processor may collect motion data. In some embodiments, the processor may collect sensor data from a sensor in the mobile device that is positioned on a specific part of a body of a mobile device user (e.g., held on or near the user's sternum, etc.) as the mobile device user walks a closed course. In some embodiments, the processor may passively collect the sensor data from a sensor in the mobile device as the user goes about his or her day and/or for a set period of time. In some embodiments, the processor may be configured to determine whether the mobile device is being held, carried, or moved by the user, and passively collect the sensor data from a sensor in the mobile device in response to determining that the mobile device is currently being held, carried, or moved by the user.

In block 604, the processor may use the collected motion data to generate a biokinetographic (BKG) waveform information structure that characterizes changes in motion data over time. The BKG waveform may be an information structure representation of the collected raw motion sensor data. In some embodiments, the processor may create the BKG waveform information structure by processing the raw motion sensor data by filtering out noise, identifying key features, and segmenting the data based on specific gait events (e.g., heel strike, toe strike, toe lift-off, cadence, etc.), computing the relevant parameters (e.g., velocity, acceleration, displacement, etc.) from the processed motion data, and mapping these parameters onto an information structure representation. In some embodiments, the BKG waveform may be a compressed and structured representation of the collected raw motion sensor data that captures the information about the user's gait patterns and their temporal evolution. This representation may enable efficient storage, analysis, and comparison of gait data, and may be used as input for further processing and machine learning algorithms to identify potential health issues, monitor progress of health condition, determine sobriety or drug compliance, evaluate the effectiveness of therapies, generate a diagnosis, etc.

In block 606, the processor may identify patterns and irregularities in the individual's gait or motion based on the BKG waveform. In some embodiments, the processor may identify patterns and irregularities in the individual's gait or motion by applying various signal processing and machine learning techniques to the BKG waveform. For example, the processor may analyze the BKG waveform to extract relevant features (e.g., amplitude, frequency, duration, phase relationships of different gait events, etc.), determine walking patterns (e.g., stride length, cadence, gait symmetry, etc.), use statistical methods (e.g., calculating mean values, standard deviations, and correlation coefficients, etc.) to identify trends and relationships among the extracted features, and/or apply machine learning algorithms (e.g., clustering, classification, or anomaly detection techniques, etc.) to compare the extracted features from the individual's BKG waveform with a database of BKG waveforms from healthy individuals or individuals with known disorders. The processor may also use other machine learning techniques, such as deep learning or recurrent neural networks, to model the temporal dynamics of the BKG waveform and capture complex patterns or irregularities.

In block 608, the processor may generate output based on the identified patterns and irregularities. In some embodiments, the output may include a diagnosis of a neuromuscular disorder. In some embodiments, the output may indicate frailty, malingering, an increased risk of falls, sobriety, drug use, drug discovery, compliance with a drug regimen, compliance with a drug regimen, etc. In some embodiments, the processor may determine quantitative metrics (e.g., gait variability indices, symmetry ratios, anomaly scores, etc.) that quantify the identified patterns and irregularities. These numerical values may be presented in tables or as summary statistics, allowing users to track their progress over time, compare their performance with others, or evaluate the effectiveness of treatments. The processor may also create plots, charts, or heatmaps that illustrate the extracted gait features, detected irregularities, compare the individual's gait patterns with those of a reference group or normative dataset, etc.

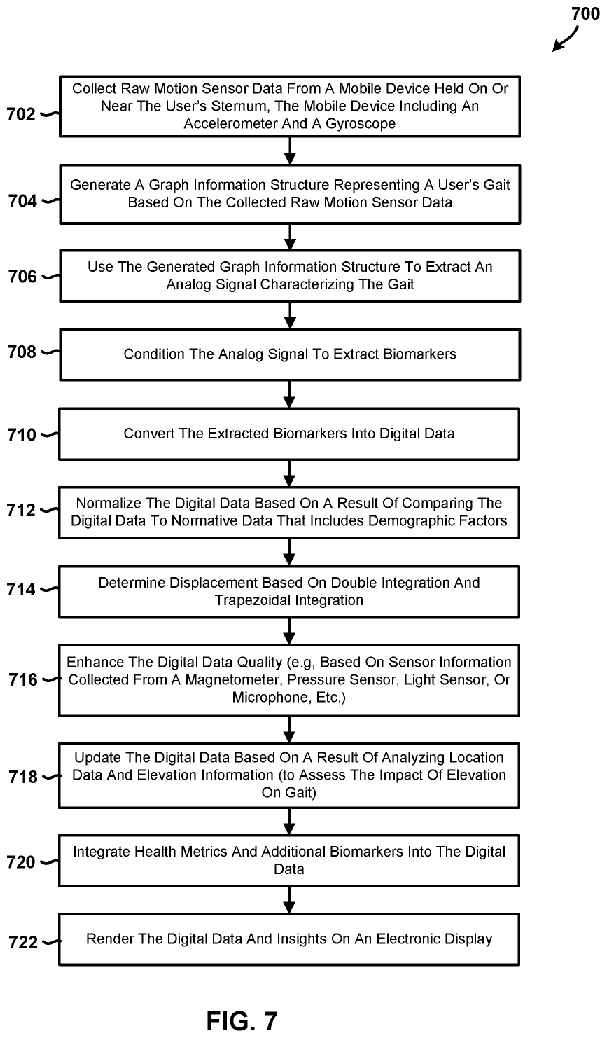

With reference to FIG. 7, in block 702, the processor may collect raw motion sensor data from a mobile device held on or near the user's sternum. In block 704, the processor may generate a graph information structure (or another information structure representation) representing a user's gait based on the collected raw motion sensor data. In block 706, the processor may use the generated graph information structure to extract an analog signal characterizing the gait. In block 708, the processor may condition the analog signal to extract biomarkers. In block 710, the processor may convert the extracted biomarkers into digital data. In block 712, the processor may normalize the digital data based on a result of comparing the digital data to normative data that includes demographic factors. In block 714, the processor may determine displacement based on double integration and trapezoidal integration. In block 716, the processor may enhance the digital data quality (e.g., based on sensor information collected from a magnetometer, pressure sensor, light sensor, or microphone, etc.). In block 718, the processor may update the digital data based on a result of analyzing location data and elevation information (to assess the impact of elevation on gait). In block 720, the processor may integrate health metrics and additional biomarkers into the digital data. In block 722, the processor may render the digital data and insights on an electronic display (e.g., by performing any or all of the operations discussed above with reference to block 608, etc.).

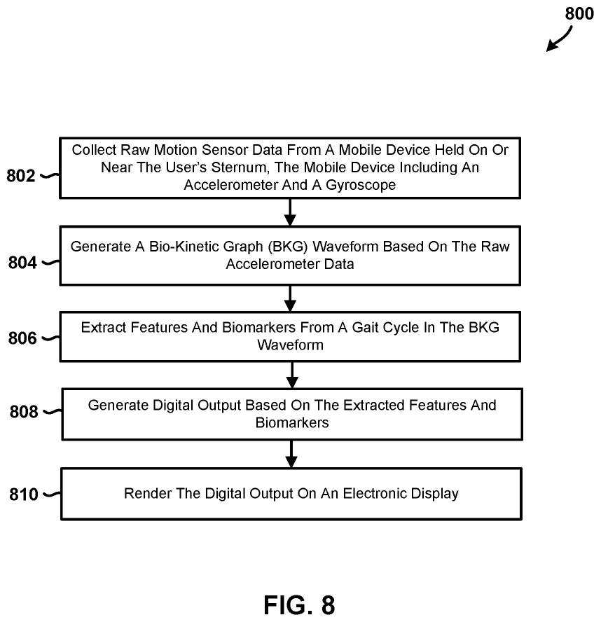

With reference to FIG. 8, in block 802, the processor may collect raw motion sensor data from a mobile device held on or near the user's sternum. In block 804, the processor may generate a bio-kinetic graph (BKG) waveform based on the raw accelerometer data. In block 806, the processor may extract features and biomarkers from a gait cycle in the BKG waveform. In block 808, the processor may generate digital output based on the extracted features and biomarkers. In block 810, the processor may render the digital output on an electronic display.

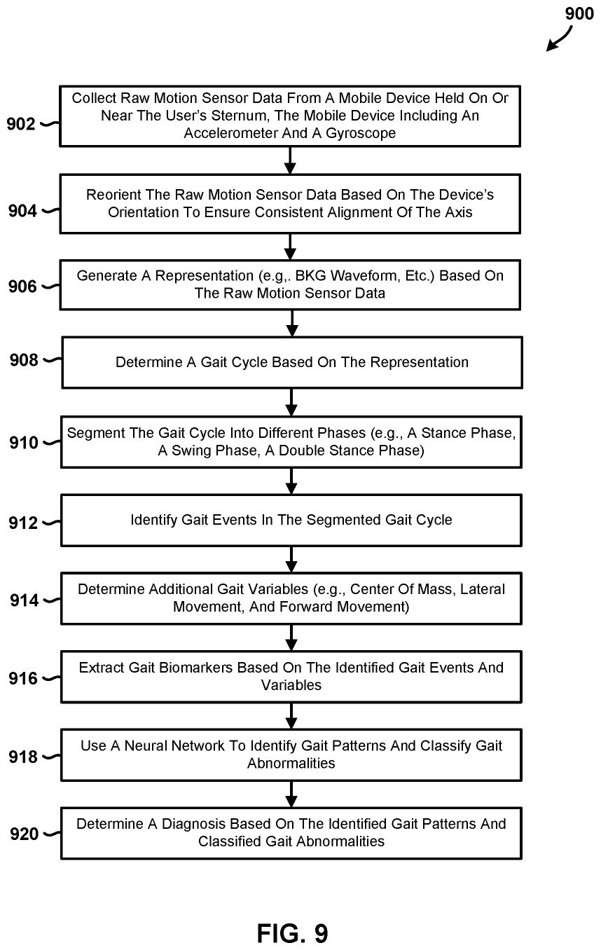

With reference to FIG. 9, in block 902, the processor may collect raw motion sensor data from a mobile device held on or near the user's sternum. In block 904, the processor may reorient the raw motion sensor data based on the device's orientation to ensure consistent alignment of the axis. In block 906, the processor may generate a representation (e.g., BKG waveform, etc.) based on the raw motion sensor data. In block 908, the processor may determine a gait cycle based on the representation. In block 910, the processor may segment the gait cycle into different phases (e.g., a stance phase, a swing phase, a double stance phase). In block 912, the processor may identify gait events in the segmented gait cycle. In block 914, the processor may determine additional gait variables (e.g., center of mass, lateral movement, and forward movement). In block 916, the processor may extract gait biomarkers based on the identified gait events and variables.

In block 918, the processor may use a neural network to identify gait patterns and classify gait abnormalities. For example, the processor may use a CNN to capture spatial patterns in the gait features, and a RNN or a long short-term memory network to model temporal dependencies in the gait data. The processor may train the neural network using a labeled dataset of gait patterns from healthy individuals and individuals with known abnormalities. The processor may use the trained network to classify the user's gait patterns based on the extracted gait features or representation (e.g., BKG waveform, etc.). The neural network may output a probability distribution over the possible gait classes, which may be used by the processor to determine the most likely gait pattern or abnormality for the user. In block 920, the processor may determine a diagnosis based on the identified gait patterns and classify gait abnormalities (or based on whether probabilities associated with the gait pattern or abnormality for the user exceed a threshold value).

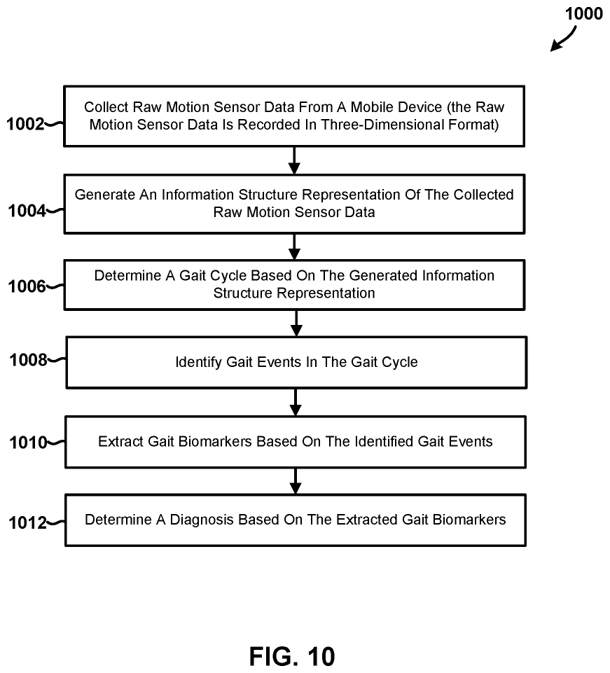

With reference to FIG. 10, in block 1002, the processor may collect raw motion sensor data from a mobile device (e.g., smartphone, wearable device, etc.). In some embodiments, the raw motion sensor data may be recorded in a three-dimensional format. In some embodiments, collecting motion sensor data from the mobile device in block 1002 may include collecting sensor data from a sensor in the mobile device that is positioned on a specific part of a body of a mobile device user (e.g., held on or near a sternum, sacrum, waist, hip, ankle, etc. of a mobile device user, etc.) as the mobile device user walks a closed course. In some embodiments, collecting motion sensor data from the mobile device in block 1002 may include collecting the raw motion sensor data from a wearable device held on or near a sternum of a mobile device user.

In some embodiments, collecting motion sensor data from the mobile device in block 1002 may include passively collecting sensor data from a sensor in the mobile device. In some embodiments, the processor may passively collect sensor data in a background process. In some embodiments, passively collecting sensor data may include the processor periodically gathering sensor data even when the user is not actively interacting with a specific application. The processor may continuously monitor the sensor readings to obtain information about the user's movements and gait patterns without requiring the user to actively initiate data collection. This passive approach may reduce the burden on the user and enable the continuous assessment of gait patterns during daily activities.

In some embodiments, the processor may passively collect sensor data by monitoring for specific events or conditions, such as a change in the user's activity state or location, which may indicate a potential gait-related event. In response to detecting such an event, the processor may activate the relevant sensors and begin collecting data. For example, the processor may initiate data collection in response to determining, based on information collected by the device's accelerometer or GPS data, that the user has started walking or running. As another example, the processor may combine data from the mobile device's GPS, accelerometer, gyroscope, and ambient light sensors to determine that the user is outdoors and walking. Such an event-driven approach may ensure that data collection is focused on relevant events. The event-driven approach may also conserve battery life (by avoiding continuous monitoring), further improving the performance and functioning of the mobile device.

In some embodiments, the processor may collect additional sensor data from one or more of a magnetometer, a pressure sensor, a light sensor, a microphone, or an infrared sensor.

In some embodiments, the processor may reorient the raw motion sensor data based on the orientation of the mobile device during the collection of the raw motion sensor data to ensure consistent alignment of an axis of three-dimensional sensor data.

In block 1004, the processor may generate an information structure representation of the collected raw motion sensor data. For example, the processor may generate a biokinetographic (BKG) waveform information structure based on the raw motion sensor data in block 1004.

In block 1006, the processor may determine a gait cycle based on the generated information structure representation. For example, the processor may identify key events (e.g., heel strike, toe strike, etc.) or turning points within the gait data that correspond to specific phases of the gait cycle and use any of the techniques discussed above (e.g., peak detection, zero-crossing analysis, or threshold-based methods, etc.) to pinpoint these key events within the information structure representation. As further examples, the processor may detect peaks in the vertical acceleration data that correspond to heel strikes, identify zero-crossings in the anterior-posterior acceleration data that coincide with mid-stance, etc. The processor may determine the duration and boundaries of a complete gait cycle based on temporal relationships between identified key events. In some embodiments, the processor may determine the duration of a gait cycle and establish its boundaries within the information structure representation by measuring the time interval between successive heel strike events or other identified key events.

In block 1008, the processor may identify gait events in the gait cycle. For example, the processor may analyze specific features and patterns in the information structure representation that correspond to the identified key events within the gait cycle. In some embodiments, the processor may use signal processing techniques and/or machine learning algorithms to detect the gait events. For example, the processor may use peak detection algorithms to identify local maxima and minima in the acceleration or velocity data that correspond to different gait events. As another example, the processor may apply threshold-based methods to detect changes in the signal that signify transitions between gait events, such as crossing a pre-defined threshold value in the vertical acceleration data. The processor may use any of the machine learning techniques discussed in this application to recognize patterns in the information structure representation that are associated with specific gait events.

In block 1010, the processor may extract gait biomarkers based on the identified gait events. For example, the processor may analyze the relationships between gait events and determine various quantitative measures that describe the user's walking patterns and biomechanics. The processor may determine gait biomarkers (e.g., stride length, step length, cadence, stance time, swing time, double support time, gait symmetry, etc.) based on the temporal and spatial relationships between the identified gait events, such as the time intervals between successive heel strikes or the distances between consecutive toe strikes. As an example, the processor may determine gait symmetry indices by comparing the gait parameters of the left and right limbs, such as the stride length or stance time of each leg. A high degree of gait symmetry may indicate a healthy walking pattern, whereas a significant asymmetry could suggest an underlying health issue or injury. In some embodiments, the processor may use a neural network to identify gait patterns and classify gait abnormalities (e.g., by performing the operations of block 918 or any of the techniques discussed in this application).

In block 1012, the processor may determine a diagnosis based on the extracted gait biomarkers. In some embodiments, the processor may determine the diagnosis based on the extracted gait biomarkers, identified gait patterns, and classified gait abnormalities. In some embodiments, the processor may determine the diagnosis based on the extracted gait biomarkers and the additional sensor data.

In some embodiments, the processor may assess, screen, monitor, diagnose, and/or prognose any of a variety of neurological, orthopedic, cardiovascular, respiratory, psychiatric and/or geriatric conditions (e.g., in any of all of blocks 418, 420, 520, 522, 606, 608, 722, 808, 810, 918, 920, and 1012).

In some embodiments, the raw motion sensor data may be collected by a wearable device (e.g., smartwatch), and the operations in blocks 1004-1012 may be performed by a processor in a smartphone.

In some embodiments, the raw motion sensor data may be collected by a wearable device (e.g., smartwatch), and the operations in blocks 1004-1012 may be performed by a processor in a server computing device. For example, in some embodiments, method 1000 may include sending the raw motion sensor data from the wearable device to the smartphone and sending the raw motion sensor data from the smartphone to the server computing device. In some embodiments, method 1000 may include sending the raw motion sensor data from the wearable device to the server computing device.

Some embodiments may include methods of analyzing a patient's gait (e.g., to identify orthopedic injuries, etc.). The method may include automatically associating multiple BKG comparison results for a unique patient with a specific dysfunction. Each comparison result may be obtained through an automatic comparison of a BKG value to a standard for a corresponding variable linked to one or more specific components of a gait cycle, including left and right heel strikes, toe strikes, and toe lift-offs. BKG values may be determined automatically from a data set consisting of numerous scalar sums of acceleration values in three orthogonal directions, with each sum corresponding to a specific point in time. The cause of the patient's condition may be identified (e.g., as an orthopedic injury, etc.) when the BKG value for the patient shows asymmetrical cadence with a difference between the first and second double stance time greater than 25 milliseconds.

In some embodiments, the method may further include obtaining the BKG data set, determining BKG values from the data set, comparing each value to the standard for the corresponding variable, and rendering the data set and comparison results. In some embodiments, the method may further include diagnosing, assessing, determining treatment for, and providing a prognosis regarding the specific dysfunction. In some embodiments, the method may further include predicting the likelihood of falling and identifying a second specific BKG pattern, which may then be associated with a second specific dysfunction and assessed. In some embodiments, the method may further include assessing an overall health status based on the plurality of BKG comparison results.

In some embodiments, the method may further include generating data for the BKG data set using various sensors adapted to sense different moving parts of a subject, a recording device to acquire motion data, a memory to store the data, and a processor to convert motion data into BKG data. The conversion of motion data into BKG data may include graphing the scalar sums of acceleration from each sensor over time.

In some embodiments, the methods may include associating multiple BKG comparison results with a specific dysfunction from a group of dysfunctions. BKG values may be determined from a data set containing scalar sums of acceleration values in three orthogonal directions, with each sum corresponding to a specific point in time. The BKG data set may be obtained, and for each variable, a value may be determined from the data set and compared to a standard for the corresponding variable. BKG data and comparison results may be used to diagnose and assess the specific dysfunction, to determine a suitable treatment and prognosis. In some embodiments, the methods may include predicting the likelihood of falling, identifying a second specific BKG pattern, associating it with a second specific dysfunction, and assessing the second dysfunction. Overall health status may also be assessed based on the BKG comparison results.

In some embodiments, data for the BKG data may be generated using various sensors adapted to sense different moving parts of a subject, a recording device to acquire motion data, a memory to store the data, and a processor to convert motion data into BKG data. In some embodiments, data for the BKG data may be generated based on sensors included in a mobile device (smartphone or wearable). In some embodiments, the method may include graphing scalar sums of acceleration from each sensor over time to convert motion data into BKG data. The subject holds, puts or positions sensors on the wrists, neck, sacrum, and/or ankles to generate motion data. The mobile device may acquire motion data wirelessly or through other remote means. Motion data may be stored in individual memory channels, and BKG data may be rendered or presented in the form of waveforms or waveform images.

Various embodiments illustrated and described are provided merely as examples to illustrate various features of the claims. However, features shown and described with respect to any given embodiment are not necessarily limited to the associated embodiment and may be used or combined with other embodiments that are shown and described. Further, the claims are not intended to be limited by any one example embodiment. For example, one or more of the operations of the methods 400, 500, 600, 700, 800, 900, 1000 may be substituted for or combined with one or more operations of the methods 400, 500, 600, 700, 800, 900, 1000, and vice versa.

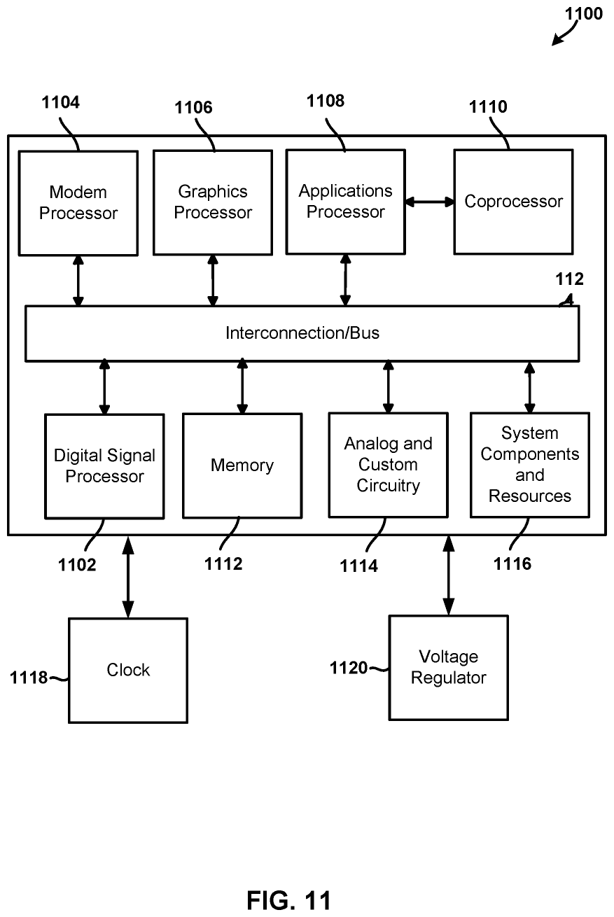

The various embodiments may be implemented on a number of multicore and multiprocessor systems, including a system-on-chip (SOC), which may include a mobile device. FIG. 11 is an architectural diagram illustrating an example SOC 1100 architecture that may be used to implement the various embodiments. The SOC 1100 may include a number of heterogeneous processors, such as a digital signal processor (DSP) 1102, a modem processor 1104, a graphics processor 1106, and an application processor 1108. The SOC 1100 may also include one or more coprocessors 1110 (e.g., vector co-processor) connected to one or more of the processors 1102, 1104, 1106, 1108. Each processor 1102, 1104, 1106, 1108, 1110 may include one or more cores, and each processor/core may perform operations independent of the other processors/cores. For example, the SOC 1100 may include a processor that executes a first type of operating system (e.g., FreeBSD, UNIX, OS X, etc.) and a processor that executes a second type of operating system (e.g., Microsoft Windows 7).

The SOC 1100 may also include analog circuitry and custom circuitry 1114 for managing sensor data, analog-to-digital conversions, wireless data transmissions, and for performing other specialized operations, such as processing encoded audio signals for games and movies. The SOC 1100 may further include system components and resources 1116, such as voltage regulators, oscillators, phase-locked loops, peripheral bridges, data controllers, memory controllers, system controllers, access ports, timers, and other similar components used to support the processors and clients running on a computing device.

The system components 1116 and custom circuitry 1114 may include circuitry to interface with peripheral devices, such as cameras, electronic displays, wireless communication devices, external memory chips, etc. The processors 1102, 1104, 1106, 1108 may be interconnected to one or more memory elements 1112, system components, and resources 1116 and custom circuitry 1114 via an interconnection/bus module 1124, which may include an array of reconfigurable logic gates and/or implement a bus architecture (e.g., CoreConnect, AMBA, etc.). Communications may be provided by advanced interconnects, such as high performance networks-on chip (NoCs).

The SOC 1100 may further include an input/output module (not illustrated) for communicating with resources external to the SOC, such as a clock 1118 and a voltage regulator 1120. Resources external to the SOC (e.g., clock 1118, voltage regulator 1120) may be shared by two or more of the internal SOC processors/cores (e.g., DSP 1102, modem processor 1104, graphics processor 1106, applications processor 1108, etc.).

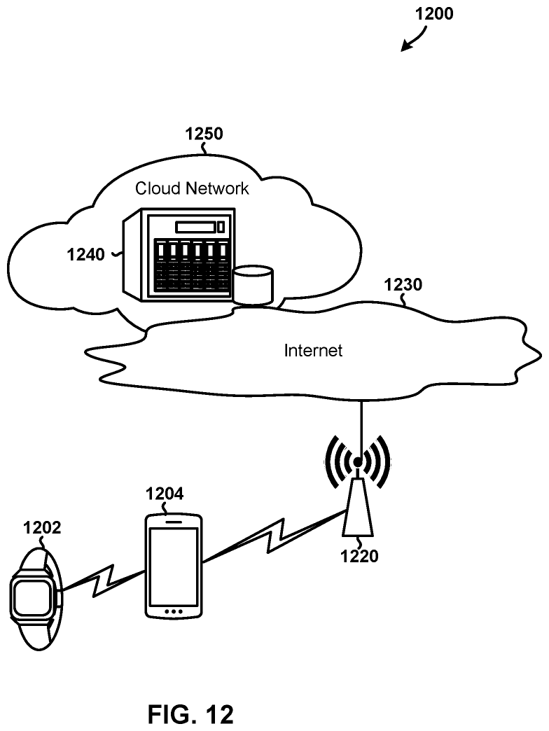

The various embodiments (including, but not limited to, embodiments described above with reference to FIGS. 1-10) may be implemented in a wide variety of computing systems, examples of which are illustrated in FIG. 12. For example, in various embodiments, the SOC 1100, any or all of the components of the computing device 102, and/or any or all operations of the methods 400, 500, 600, 700, 800, 900, 1000 may be performed in a wearable device 1202, in a smartphone 1204, or server computing device 1250, which may be deployed in a cloud network 1250.

In particular, the system 1200 illustrated in FIG. 12 includes a wearable device 1202, a smartphone 1204, and a server computing device 1250 coupled together via wireless communication links. The wearable device 1202 may be coupled to the smartphone 1204 via direct and indirect wireless communication links (e.g., home router, Bluetooth, etc.). The wearable device 1202 and smartphone 1204 may be coupled to a customer premise equipment (CPE) 1220 component/device via wired and wireless communication links. The CPE 1220 may include communication links to a service provider network and wide area network (WAN) that allow the smartphone 1204 to send and receive information to and from the Internet and ultimately to the server computing device 1250 in the cloud network 1250.

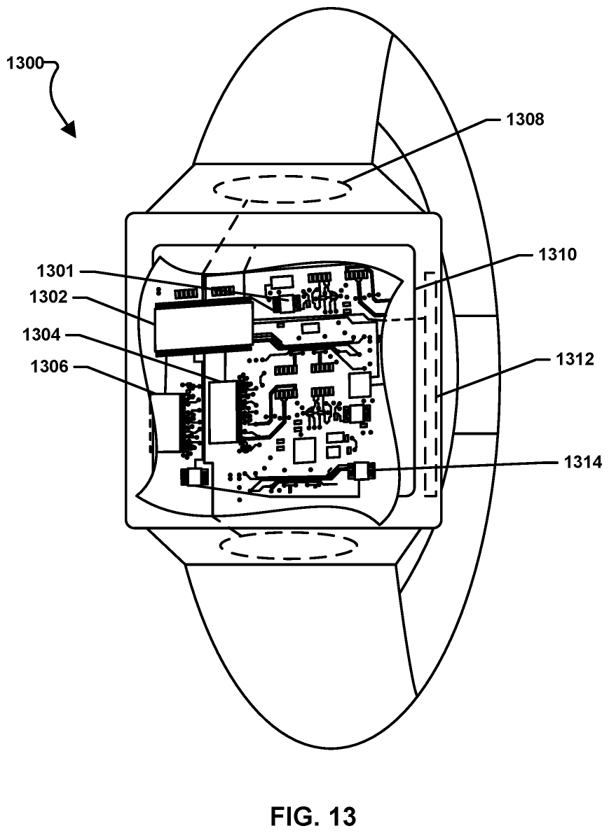

The various embodiments may be implemented within a variety of computing devices, such as a wearable computing device. FIG. 13 illustrates an example wearable computing device (e.g., device 1202, etc.) in the form of a smart watch 1300 according to some embodiments. A smartwatch 1300 may include a processor 1301 and/or an SoC 1302 including two or more processors (e.g., application processor, low power processor) coupled to internal memories 1304 and 1306. Internal memories 1304, 1306 may be volatile or non-volatile memories, and may also be secure and/or encrypted memories, or unsecured and/or unencrypted memories, or any combination thereof. The SoC 1302 may also be coupled to a touchscreen display 1310, such as a resistive-sensing touchscreen, capacitive-sensing touchscreen infrared sensing touchscreen, or the like. Additionally, the smart watch 1300 may have one or more antenna 1308 for sending and receiving electromagnetic radiation that may be connected to one or more wireless data links, such as one or more Bluetooth® transceivers, Peanut transceivers, Wi-Fi transceivers, ANT+ transceivers, etc., which may be coupled to the SoC 1302. The smart watch 1300 may also include physical or virtual buttons for receiving user inputs as well as a slide sensor 1312 for receiving user inputs. The smartwatch 1300 may also include any of a variety of sensors 1314, including any or all of the sensers discussed in the application.

The touchscreen display 1310 may be coupled to a touchscreen interface module that is configured receive signals from the touchscreen display 1310 indicative of locations on the screen where a user's fingertip or a stylus is touching the surface and output to the SoC 1302 information regarding the coordinates of touch events. Further, the SoC 1302 may be configured with processor-executable instructions to correlate images presented on the touchscreen display 1310 with the location of touch events received from the touchscreen interface module in order to detect when a user has interacted with a graphical interface icon, such as a virtual button.

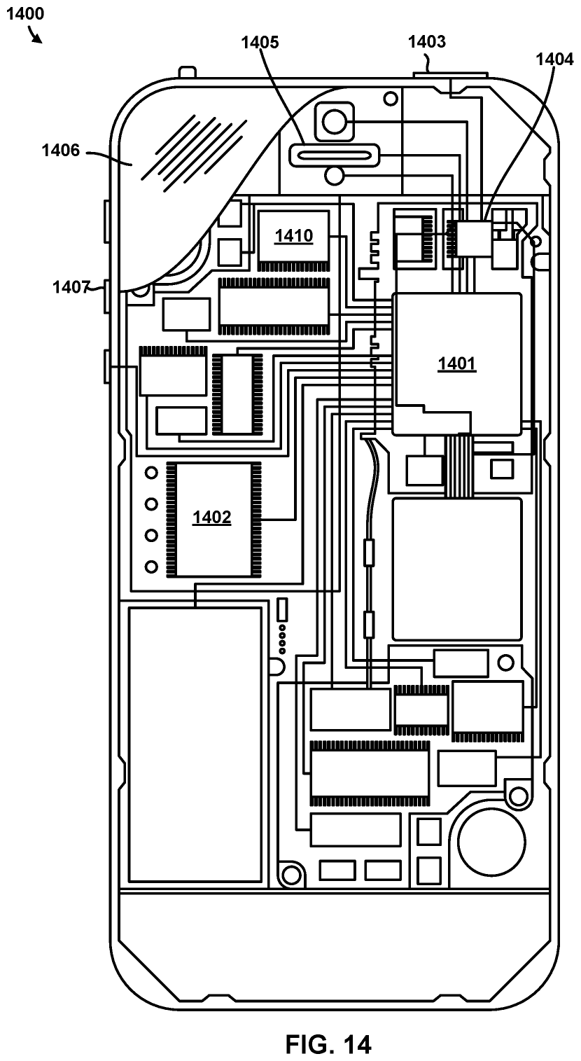

FIG. 14 is a component block diagram of a computing device (e.g., computing device 1204, etc.) suitable for use with various embodiments. With reference to FIGS. 1-14, various embodiments may be implemented on a variety of computing devices 1400 (e.g., 102, 1104, etc.), an example of which is illustrated in FIG. 14 in the form of a smartphone 1400. The smartphone 1400 may include a processor 1401 or SOC coupled to internal memory 1402, a display 1406, and to a speaker 1405. The processor 1401 may also be coupled to at least one SIM 1410 and/or a SIM interface that may store information supporting a first 5GNR subscription and a second 5GNR subscription, which support service on a 5G non-standalone (NSA) network.

The smartphone 1400 may include an antenna 1403 for sending and receiving electromagnetic radiation that may be connected to a wireless transceiver 1404 coupled to one or more processors 1401. The smartphone 1400 may also include menu selection buttons or rocker switches 1407 for receiving user inputs.

The smartphone 1400 may also include a sound encoding/decoding (CODEC) circuit, which digitizes sound received from a microphone into data packets suitable for wireless transmission and decodes received sound data packets to generate analog signals that are provided to the speaker to generate sound. Also, one or more of the processors, wireless transceiver and CODEC may include a digital signal processor (DSP) circuit (not shown separately).

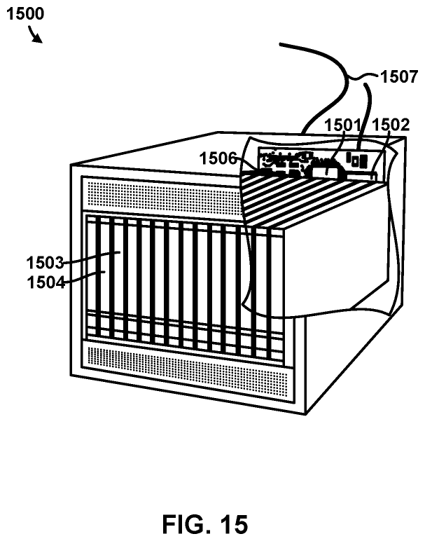

Some embodiments (e.g., server 1240, etc.) may be implemented on any of a variety of commercially available computing devices, such as the server computing device 1500 illustrated in FIG. 15. Such a server device 1500 may include a processor 1501 coupled to volatile memory 1502 and a large capacity nonvolatile memory, such as a disk drive 1503. The server device 1500 may also include a floppy disc drive, USB, etc. coupled to the processor 1501. The server device 1500 may also include network access ports 1506 coupled to the processor 1501 for establishing data connections with a network connection circuit 1504 and a communication network (e.g., IP network) coupled to other communication system network elements.

The processors discussed in this application may be any programmable microprocessor, microcomputer or multiple processor chip or chips that can be configured by software instructions (applications) to perform a variety of functions, including the functions of the various embodiments described above. In some devices, multiple processors may be provided, such as one processor dedicated to wireless communication functions and one processor dedicated to running other applications. Typically, software applications may be stored in the internal memory before they are accessed and loaded into the processors. The processors may include internal memory sufficient to store the application software instructions. In many devices, the internal memory may be a volatile or nonvolatile memory, such as flash memory, or a mixture of both. For the purposes of this description, a general reference to memory refers to memory accessible by the processors including internal memory or removable memory plugged into the device and memory within the processors themselves. Additionally, as used herein, any reference to a memory may be a reference to a memory storage and the terms may be used interchangeably.

Implementation examples are described in the following paragraphs. While some of the following implementation examples are described in terms of example methods, further example implementations may include: the example methods discussed in the following paragraphs implemented by a computing device including a processor configured with processor-executable instructions to perform operations of the methods of the following implementation examples; the example methods discussed in the following paragraphs implemented by a computing device including means for performing functions of the methods of the following implementation examples; and the example methods discussed in the following paragraphs may be implemented as a non-transitory processor-readable storage medium having stored thereon processor-executable instructions configured to cause a processor of a computing device to perform the operations of the methods of the following implementation examples.

Example 1: A method of representing human gait to diagnose health conditions, including collecting raw motion sensor data from a mobile device, in which the raw motion sensor data is recorded in three-dimensional format, generating an information structure representation of the collected raw motion sensor data, determining a gait cycle based on the generated information structure representation, identifying gait events in the gait cycle, extracting gait biomarkers based on the identified gait events, and determining a diagnosis based on the extracted gait biomarkers.

Example 2: The method of example 1, in which generating the information structure representation of the collected raw motion sensor data includes generating a biokinetographic (BKG) waveform information structure based on the raw motion sensor data.

Example 3: The method of any of the examples 1 and 2, in which collecting motion sensor data from the mobile device includes collecting sensor data from a sensor in the mobile device that is positioned on a specific part of a body of a mobile device user as the mobile device user walks a closed course.

Example 4: The method of any of the examples 1 and 2, in which collecting motion sensor data from the mobile device includes passively collecting sensor data from a sensor in the mobile device.

Example 5: The method of any of the examples 1-4, further including reorienting the raw motion sensor data based on an orientation of the mobile device during collection of the raw motion sensor data to ensure consistent alignment of an axis of three-dimensional sensor data.

Example 6: The method of any of the examples 1-5, further including using a neural network to identify gait patterns and classify gait abnormalities, in which determining the diagnosis based on the extracted gait biomarkers includes determining the diagnosis based on the extracted gait biomarkers, identified gait patterns, and classified gait abnormalities.

Example 7: The method of any of the examples 1-6, further including collecting additional sensor data from one or more of a magnetometer, a pressure sensor, a light sensor, a microphone, or an infrared sensor, in which determining the diagnosis based on the extracted gait biomarkers includes determining the diagnosis based on the extracted gait biomarkers and the additional sensor data.

Example 8: The method of any of the examples 1-7, in which the mobile device is a smartphone.

Example 9: The method of any of the examples 1-7, in which collecting motion sensor data from the mobile device includes collecting the raw motion sensor data from a wearable device held on or near a sternum of a mobile device user, and the operations of generating the information structure representation of the collected raw motion sensor data, determining the gait cycle based on the generated information structure representation, identifying the gait events in the gait cycle, extracting the gait biomarkers based on the identified gait events, and determining the diagnosis based on the extracted gait biomarkers are performed on at least one of a smartphone or server computing device.

Example 10: The method of example 9, further including sending the raw motion sensor data from the wearable device to the smartphone and sending the raw motion sensor data from the smartphone to the server computing device.

The foregoing method descriptions and the process flow diagrams are provided merely as illustrative examples and are not intended to require or imply that the steps of the various embodiments must be performed in the order presented. As will be appreciated by one of skill in the art the order of steps in the foregoing embodiments may be performed in any order. Words such as ‘thereafter,’ ‘then,’ ‘next,’ etc. are not intended to limit the order of the steps; these words are simply used to guide the reader through the description of the methods. Further, any reference to claim elements in the singular, for example, using the articles ‘a,’ ‘an’ or ‘the’ is not to be construed as limiting the element to the singular.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The hardware used to implement the various illustrative logics, logical blocks, modules, components, and circuits described in connection with the embodiments disclosed herein may be implemented or performed with a general-purpose processor, a digital signal processor (DSP), an application-specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Alternatively, some steps or methods may be performed by circuitry that is specific to a given function.

In one or more exemplary embodiments, the functions described may be implemented in hardware, software, firmware, or any combination thereof. If implemented in software, the functions may be stored as one or more instructions or code on a non-transitory computer-readable medium or non-transitory processor-readable medium. The steps of a method or algorithm disclosed herein may be embodied in a processor-executable software module and/or processor-executable instructions, which may reside on a non-transitory computer-readable or non-transitory processor-readable storage medium. Non-transitory server-readable, computer-readable or processor-readable storage media may be any storage media that may be accessed by a computer or a processor. By way of example but not limitation, such non-transitory server-readable, computer-readable or processor-readable media may include RAM, ROM, EEPROM, FLASH memory, CD-ROM or other optical disk storage, magnetic disk storage or other magnetic storage devices, or any other medium that may be used to store desired program code in the form of instructions or data structures and that may be accessed by a computer. Disk and disc, as used herein, includes compact disc (CD), laser disc, optical disc, DVD, floppy disk, and Blu-ray disc where disks usually reproduce data magnetically, while discs reproduce data optically with lasers. Combinations of the above are also included within the scope of non-transitory server-readable, computer-readable and processor-readable media. Additionally, the operations of a method or algorithm may reside as one or any combination or set of codes and/or instructions on a non-transitory server-readable, processor-readable medium and/or computer-readable medium, which may be incorporated into a computer program product.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the following claims and the principles and novel features disclosed herein.

What is claimed is:

1. A method of representing human gait to diagnose health conditions, comprising:
collecting raw motion sensor data from a mobile device, wherein the raw motion sensor data is recorded in three-dimensional format;
generating an information structure representation of the collected raw motion sensor data;
determining a gait cycle based on the generated information structure representation;
identifying gait events in the gait cycle;
extracting gait biomarkers based on the identified gait events; and
determining a diagnosis based on the extracted gait biomarkers.

2. The method of claim 1, wherein generating the information structure representation of the collected raw motion sensor data comprises generating a biokinetographic (BKG) waveform information structure based on the raw motion sensor data.

3. The method of claim 1, wherein collecting motion sensor data from the mobile device comprises collecting sensor data from a sensor in the mobile device that is positioned on a specific part of a body of a mobile device user as the mobile device user walks a closed course.

4. The method of claim 1, wherein collecting motion sensor data from the mobile device comprises passively collecting sensor data from a sensor in the mobile device.

5. The method of claim 1, further comprising reorienting the raw motion sensor data based on an orientation of the mobile device during collection of the raw motion sensor data to ensure consistent alignment of an axis of three-dimensional sensor data.

6. The method of claim 1, further comprising using a neural network to identify gait patterns and classify gait abnormalities, wherein determining the diagnosis based on the extracted gait biomarkers comprises determining the diagnosis based on the extracted gait biomarkers, identified gait patterns, and classified gait abnormalities.

7. The method of claim 1, further comprising:
collecting additional sensor data from one or more of a magnetometer, a pressure sensor, a light sensor, a microphone, or an infrared sensor,
wherein determining the diagnosis based on the extracted gait biomarkers comprises determining the diagnosis based on the extracted gait biomarkers and the additional sensor data.

8. The method of claim 1, wherein the mobile device is a smartphone.

9. The method of claim 1, wherein:
collecting motion sensor data from the mobile device comprises collecting the raw motion sensor data from a wearable device held on or near a sternum of a mobile device user; and
the operations of generating the information structure representation of the collected raw motion sensor data, determining the gait cycle based on the generated information structure representation, identifying the gait events in the gait cycle, extracting the gait biomarkers based on the identified gait events, and determining the diagnosis based on the extracted gait biomarkers are performed on at least one of a smartphone or server computing device.

10. The method of claim 9, further comprising:
sending the raw motion sensor data from the wearable device to the smartphone; and
sending the raw motion sensor data from the smartphone to the server computing device.

11. A mobile device, comprising:
a processor configured to:
collect or receive raw motion sensor data;
record the raw motion sensor data in three-dimensional format;
generate an information structure representation of the collected raw motion sensor data;
determine a gait cycle based on the generated information structure representation;
identify gait events in the gait cycle;
extract gait biomarkers based on the identified gait events; and
determine a diagnosis based on the extracted gait biomarkers.

12. The mobile device of claim 1, wherein the processor is configured to generate the information structure representation of the collected raw motion sensor data by generating a biokinetographic (BKG) waveform information structure based on the raw motion sensor data.

13. The mobile device of claim 1, wherein the processor is configured to collect motion sensor data from the mobile device by collecting sensor data from a sensor in the mobile device that is positioned on a specific part of a body of a mobile device user as the mobile device user walks a closed course.

14. The mobile device of claim 1, wherein the processor is configured to collect motion sensor data from the mobile device by passively collecting sensor data from a sensor in the mobile device.

15. The mobile device of claim 1, wherein the processor is further configured to reorient the raw motion sensor data based on an orientation of the mobile device during collection of the raw motion sensor data to ensure consistent alignment of an axis of three-dimensional sensor data.

16. The mobile device of claim 1, wherein:
the processor is further configured to use a neural network to identify gait patterns and classify gait abnormalities and
wherein the processor is configured to determining the diagnosis based on the extracted gait biomarkers by determining the diagnosis based on the extracted gait biomarkers, identified gait patterns, and classified gait abnormalities.

17. The mobile device of claim 1, wherein:
the processor is further configured to collect additional sensor data from one or more of a magnetometer, a pressure sensor, a light sensor, a microphone, or an infrared sensor; and
the processor is configured to determine the diagnosis based on the extracted gait biomarkers comprises determining the diagnosis based on the extracted gait biomarkers and the additional sensor data.

18. The mobile device of claim 11, wherein the mobile device is one of a smartphone or a wearable device.

19. A non-transitory computer readable storage medium having stored thereon processor-executable software instructions configured to cause a processor in a mobile device to perform operations for representing human gait to diagnose health conditions, the operations comprising:
collecting raw motion sensor data from the mobile device, wherein the raw motion sensor data is recorded in three-dimensional format;
generating an information structure representation of the collected raw motion sensor data;
determining a gait cycle based on the generated information structure representation;
identifying gait events in the gait cycle;
extracting gait biomarkers based on the identified gait events; and
determining a diagnosis based on the extracted gait biomarkers.

20. The non-transitory computer readable storage medium of claim 19, wherein the stored processor-executable software instructions are configured to cause a processor to perform operations such that generating the information structure representation of the collected raw motion sensor data comprises generating a biokinetographic (BKG) waveform information structure based on the raw motion sensor data.
